(19) United States
(12) Patent Application Publication
(10) Pub. No.: US 2021/0251949 A1
(43) Pub. Date: Aug. 19, 2021

(54) Compositions and Methods for Treatment of Narcolepsy and Related Disorders

(71) Applicant: California Cannabinoids, Oakland, CA (US)

(72) Inventor: David Lampach, Berkeley, CA (US)

(73) Assignee: California Cannabinoids, Berkeley, CA (US)

(21) Appl. No.: 17/252,601

(22) PCT Filed: Jun. 17, 2019
(86) PCT No.: PCT/US19/37561

§ 371 Date: Dec. 15, 2020

Related U.S. Application Data
(60) Provisional application No. 62/685,903, filed on Jun. 15, 2018.

(51) Int. Cl. A61K 31/352; A61K 31/05; A61K 9/00

(52) U.S. Cl. CPC A61K 31/352; A61K 31/05; A61K 9/0073; A61K 2236/37; A61K 2236/33; A61K 2236/39; A61K 2236/35

(57) ABSTRACT

The present disclosure describes methods and compositions for treatment for neurological conditions of narcolepsy, isolated cataplexy, sleep apnea, and like conditions. More particularly the disclosure relates to treating individuals suffering from narcolepsy and/or cataplexy, and sleep apnea with a therapeutically effective dose of one or more cannabis flower, cannabinoids, terpenes, cannabis plant extracts, cannabis compositions, or mixtures thereof.

### FIELD OF THE INVENTION

The present disclosure generally relates to the field of medicine, and to compositions and methods for treatment for neurological conditions of narcolepsy, isolated cataplexy, sleep apnea, and related conditions. More particularly, the disclosure relates to treating individuals suffering from narcolepsy and/or cataplexy, and sleep apnea with a therapeutically effective dose of one or more cannabis flower, cannabinoids, terpenes, cannabis plant extracts, cannabis compositions, or mixtures thereof.

### BACKGROUND

In the art of medicine synthetic drugs are designed to treat certain conditions. In some instances, there are naturally occurring organic compounds that may compete with synthetic drugs relative to the treatment of symptoms for certain neurological or immunological diseases or conditions such as narcolepsy. Narcolepsy is a disabling neurological disorder that was first recognized nearly 150 years ago by Gelineau, J. B. (De la narcolapsie, Gazette des Hopitaux Paris (1880) 53: 626-628).

Typical symptoms of narcolepsy are excessive daytime sleepiness (EDS), cataplexy, sleep paralysis, and hallucinations. All individuals with narcolepsy experience EDS, which is characterized by persistent sleepiness, regardless of how much sleep an individual gets at night. Other symptomatic sub-conditions, such as cataplexy, characterized by a sudden but reversible loss of muscle tone, and sleep paralysis, characterized as an inability to move at sleep onset or awakening, are present in approximately 10 to 25 percent of affected individuals during the course of their illness. In some cases, sleep paralysis is accompanied by hallucinations; hypnagogic hallucinations (during transition into a sleep state) and hypnopompic hallucinations (during transition out of a sleep state). Also, rapid eye movement (REM) sleep phase may occur at the onset of sleep in some narcoleptic persons.

Narcolepsy is classified into two types. Narcolepsy with cataplexy (ICD G47.411) is sometimes called “Type 1 narcolepsy”. Narcolepsy without cataplexy (ICD G47.419) is called “Type 2 narcolepsy”.

Sleep apnea, also known as obstructive sleep apnea (OSA) (ICD G47.33) is characterized by cessation of breathing for short periods during sleep resulting in oxygen desaturation. These pauses can occur 20-30 times per hour, and while risk factors include male gender, age, lifestyle, and obesity. It is estimated that the disorder affects 22 million Americans of a range of ages, weights and gender, with 80 percent of the cases of moderate and severe obstructive sleep apnea undiagnosed. Symptoms related to OSA include excessive daytime sleepiness, which causes an increased risk for car crashes and work-related accidents in addition for higher risk for cognitive disturbances and high blood pressure.

Narcolepsy affects an estimated 1 in every 2,000 people in the United States. That's 200,000 Americans, and approximately 3 million worldwide. There is no cure for narcolepsy, but it can be treated with long-term management of symptoms (Fry, J., Neurology (1998) 50(2 Suppl 1): S8-15). Interventions can be non-pharmacological, such as lifestyle changes, and pharmacological, for relief of daytime sleepiness, cataplexy, sleep paralysis, and/or hallucinations. Narcolepsy and cataplexy are classified as separate indications by the U.S. Food and Drug Administration (FDA). However, the classification of separate indication does not necessarily imply a separate basis of disease.

Stimulants are often prescribed for narcolepsy to increase wakefulness or to reduce the number and severity of cataplectic attacks or hypnagogic hallucinations being experienced by a patient. Modafinil, marketed as Provigil®, and similar stimulants, are typically the first line of treatment. They are selected over older amphetamine-like stimulants because they are less addictive and extremely high doses of amphetamine-like stimulants are necessary to restore alertness to normal levels (Mitler, M. et al, Sleep (1993) 16: 306-317). Such doses can have very dangerous side effects. Consequently, most doctors only prescribe amphetamine-like stimulants such as methylphenidate, Ritalin® or Adderall® to manage narcolepsy where modafinil is not effective. The amphetamine-like stimulants must be carefully monitored because they can have potentially severe side effects, such as irritability and nervousness, shakiness, disturbances in heart rhythm, and nighttime sleep disruption. In addition, health care professionals should be careful prescribing them because the potential for abuse is high with any amphetamine. Because of side effects, most narcoleptics use stimulants only when absolutely needed or continuous use of low-level doses does not restore normal levels of alertness. Periodic “drug holidays” may also be required to maintain the effectiveness of stimulants (Miller, M. S. Sleep (1994) 17: S103-S106).

Antidepressants are also used to treat Type 1 narcolepsy, and more specifically cataplexy. Cataplexy can sometimes be treated successfully with tricyclic antidepressants (including imipramine, desipramine, clomipramine, and protriptyline) or selective serotonin and noradrenergic reuptake inhibitors (including venlafaxine, fluoxetine, and atomoxetine). Both tricyclic antidepressant drugs and reuptake inhibitors all appear to act by producing metabolites that activate noradrenergic receptors. However, troublesome side effects still occur in some individuals, including impotence, high blood pressure, and heart rhythm irregularities.

Sodium oxybate (also known as gamma hydroxybutyrate or GHB, the date-rape drug) has been approved by the FDA to treat cataplexy and excessive daytime sleepiness in individuals with narcolepsy. It is a strong sedative that must be taken twice a night. However, due to the apparent safety concerns associated with the use of this drug, the distribution of sodium oxybate is tightly restricted.

A challenge to the treatment of narcolepsy with synthetic drugs is the specter of side effects, some of which can be debilitating or fatal. Moreover, synthetic drugs may provide symptom relief but obfuscate healthy amounts of sleep a patient could receive otherwise, which may lead to further setbacks in overall health of an individual.

Treatments for sleep apnea typically include surgical interventions, Continuous Positive Airway Pressure (CPAP) machines and some pharmacological treatments such as fluoxetine (an anti-depressant) and modafinil (an amphetamine-like compound) (though only modafinil has been approved by the FDA for use in patients that have residual daytime sleepiness despite optimal use of CPAP). Thus, challenges to treating sleep apnea still exist.

Therefore, what is needed in the art are new compositions and methods for treating, preventing or ameliorating the effects of Type 1 narcolepsy, Type 2 narcolepsy, sleep apnea, and related conditions, that are more effective and easier to manage. This application teaches cannabis strains, compounds and/or compositions derived from one or a combination of cannabis strains, and isolated cannabinoids for the treatment of Type 1 narcolepsy, Type 2 narcolepsy, sleep apnea, and related conditions.

### SUMMARY

The present application provides cannabis compounds, cannabis extracts, compositions comprising cannabis compounds, and methods for using the same to treat, prevent and/or ameliorate the effects of the conditions such narcolepsy, cataplexy, and related disorders known and unknown, including but not limited to sleep apnea, circadian rhythm sleep disorders and restless leg syndrome.

In any embodiment, cannabis compositions for treatment of narcolepsy may comprise raw cannabis flower, cannabis extracts, one or more cannabis compounds isolated from cannabis plants, one or more synthetic cannabis compounds, cannabis extracts fortified with one or more isolated cannabis compounds, cannabis extracts fortified with one or more isolated cannabis compounds, cannabis extracts fortified with one or more synthetic cannabis compound, or any mixture thereof.

The present application relates to cannabis compositions, which may comprise cannabis flower, extracts from a cannabis plant, one or more cannabinoid extracted and isolated from a cannabis plant, one or more terpene extracted and isolated from a cannabis plant, synthetically manufactured cannabinoids, synthetically manufactured terpenes, or any combination thereof.

Cannabis compositions, cannabis flower, and cannabis extracts described herein, comprise specific desired cannabis profiles having surprisingly high efficacy in treatment of symptoms of narcolepsy, isolated cataplexy, sleep apnea, and related disorders, known and unknown, including but not limited to circadian rhythm sleep disorders and restless leg syndrome.

Said cannabis compositions, cannabis flower, and cannabis extracts and methods of treatment to a patient may provide temporary or long-term relief from symptoms of narcolepsy, isolated cataplexy, sleep apnea, and related disorders known and unknown, including but not limited to circadian rhythm sleep disorders and restless leg syndrome.

In some embodiments, a cannabis extract may comprise Δ⁹ tetrahydrocannabinol (THC) and Δ⁹ tetrahydrocannabivarin (THCV) in desired synergistic ratios by weight as described hereinafter to achieve a prophylactic or therapeutic effect in patients.

In some embodiments, a cannabis extract may comprise Δ⁹ tetrahydrocannabinolic acid (THCA) and Δ⁹ tetrahydrocannabivarinic acid (THCVA) in desired synergistic ratios by weight as described hereinafter to achieve a prophylactic or therapeutic effect in patients.

In some embodiments a cannabis extract may comprise THC, THCA, THCV and THCVA in desired synergistic ratios by weight as described hereinafter to achieve a prophylactic or therapeutic effect in patients.

In some embodiments, a cannabis extract may comprise substantially pure mixture of THCV and THCVA in desired synergistic ratios by weight as described hereinafter to achieve a prophylactic or therapeutic effect in patients, and in a preferred embodiment said mixture of THCV and THCVA and/or said extract are substantially free of other cannabinoids.

In some embodiments, a cannabis extract may comprise substantially pure THCV in a therapeutically effective amount to achieve a prophylactic or therapeutic effect in patients, and in a preferred embodiment said extract is substantially free of other cannabinoids.

In some embodiments, a cannabis extract may comprise substantially pure THCVA in a therapeutically effective amount to achieve a prophylactic or therapeutic effect in patients, and in a preferred embodiment said extract is substantially free of other cannabinoids.

In a preferred embodiment, a cannabis extract or raw cannabis flower has a ratio of THC to THCV, wherein said cannabis extract or raw cannabis flower may be used to produce a cannabis composition having a range of the ratio of THC to THCV from approximately 25% to 75% to approximately 75% to 25%.

In a preferred embodiment, a cannabis extract or raw cannabis flower has a ratio of THCA to THCVA, wherein said cannabis extract or raw cannabis flower may be used to produce a cannabis composition having a range of the ratio of THCA to THCVA from approximately 25% to 75% to approximately 75% to 25%.

In a preferred embodiment, a cannabis extract or raw cannabis flower has a ratio of THC, THCA, THCV to THCVA, wherein said cannabis extract or raw cannabis flower may be used to produce a cannabis composition having a range of the ratio of THC to THCV from approximately 25% to 75% to approximately 75% to 25%.

In a preferred embodiment, a cannabis extract or raw cannabis flower has a ratio of THCA to THCVA, wherein said cannabis extract or raw cannabis flower may be used to produce a cannabis composition having a range of the ratio of THC to THCV from approximately from approximately 25% to 75% to approximately 75% to 25%.

In a preferred embodiment, a cannabis extract or raw cannabis flower has a ratio of THCA to THCVA, wherein said cannabis extract or raw cannabis flower may be used to produce a cannabis composition having a range of the ratio of a mixture of THC and THCA to a mixture of THCV and THCVA from approximately from approximately 25% to 75% to approximately 75% to 25%.

In a preferred embodiment, a cannabis extract or raw cannabis flower has a ratio of THC, THCA, THCV to THCVA, wherein said cannabis extract or raw cannabis flower may be used to produce a cannabis composition having a range of the ratio of a mixture of THC and

THCA to a mixture of THCV and THCVA from approximately from approximately 25% to 75% to approximately 75% to 25%

In any embodiment a cannabis extract or cannabis composition may further comprise β-caryophyllene in desired synergistic ratios with the desired cannabinoids by weight as described hereinafter to achieve a prophylactic or therapeutic effect in patients.

In some embodiments, the cannabis extract of the present invention may be collected from the cannabis plant, plant part, tissue, or cell of a cannabis plant described herein.

In some embodiments, the cannabis extract of the present invention is selected from the group consisting of kief, hashish, bubble hash, solvent reduced oils, sludges, e-juice, and tinctures.

In some embodiments, the cannabis extract of the present invention retains the terpene profile of the cannabis plant, plant part, tissue or cell from which it was made.

In some embodiments, the cannabis extract of the present invention substantially retains the terpene profile of the cannabis plant, plant part, tissue or cell from which it was made.

In some embodiments, the cannabis extract of the present invention retains the cannabis profile of the cannabis plant, plant part, tissue or cell from which it was made.

In some embodiments, the cannabis extract of the present invention substantially retains the cannabis profile of the cannabis plant, plant part, tissue or cell from which it was made.

In any embodiment, a cannabis extract may be comprised of 100% naturally occurring cannabinoids and/or terpenes extracted from the group consisting of one or more cannabis plants, cannabis plant parts or raw cannabis flowers.

In any embodiment, a cannabis extract may be comprised of 100% synthetically made cannabinoids and/or terpenes.

In any embodiment, a cannabis extract may be comprised of any mixture of synthetically made cannabinoids and terpenes, and cannabinoids and/or terpenes extracted from the group consisting of one or more cannabis plants, cannabis plant parts or raw cannabis flowers.

In any embodiment described herein, a cannabis composition may consist of one cannabis extract described herein.

In any embodiment described herein, a cannabis composition may comprise one or more cannabis extracts described herein.

In any embodiment described herein, a cannabis composition may consist of two or more cannabis extracts described herein.

In any embodiment described herein, a cannabis composition may comprise one or more cannabis extracts described herein, and a pharmaceutically acceptable carrier known to one of skill in the art.

In any embodiment described herein, a cannabis composition, may further comprise (β-caryophyllene in desired synergistic ratios with the desired cannabinoids by weight as described hereinafter to achieve a prophylactic or therapeutic effect in patients.

In some embodiments, a cannabis edible product is produced from the cannabis plant, plant part, tissue, or cell.

In some embodiments, a cannabis edible product is produced from one or more cannabis extracts described herein.

In some embodiments, a cannabis edible product is produced from one or more cannabis plants, plant parts, tissues, or cells.

In some embodiments, a cannabis transdermal delivery via a patch is produced from one or more cannabis plants, plant parts, tissues, or cells.

In some embodiments, a cannabis transdermal delivery via a patch is produced from one or more cannabis extracts described herein.

In some embodiments, a cannabis transdermal delivery via a patch is produced from one or more cannabis compositions described herein.

In some embodiments, the present application teaches a cannabis tincture for smoking or vaporization, wherein the tincture comprises one or more cannabis compositions described herein.

In some embodiments, the present application teaches a cannabis tincture for smoking or vaporization, wherein the tincture comprises one or more cannabis extracts described herein.

In some embodiments, the present application teaches a compressed cannabis pellet for smoking or vaporization, wherein the pellet comprises one or more of the cannabis compositions described herein.

In some embodiments, the present application teaches a compressed cannabis pellet for smoking or vaporization, wherein the pellet comprises one or more of the cannabis extracts described herein.

In some embodiments, the compressed cannabis pellet of the present application comprises one or more cannabis extract described herein.

In some embodiments, the compressed cannabis pellet of the present application comprises one or more cannabis extract described herein.

In some embodiments, the compressed cannabis pellet of the present application is in the shape of a truncated cone.

In some embodiments, the compressed cannabis pellet of the present application is a truncated cone, with a height of 2.0 millimeters, a smaller base diameter of 4.0 millimeters, and a larger base diameter of 6.0 millimeters.

In some embodiments, the compressed cannabis pellet of the present application is in the shape of a donut.

In some embodiments, the compressed cannabis pellet of the present application is a donut shape with a height of 2.0 millimeters, an inner donut diameter of 1.5 millimeters, and an outer donut diameter of 6 millimeters.

In some embodiments, the present invention teaches a method of treating Type 1 narcolepsy, said method comprising: (i) identifying a patient with Type 1 narcolepsy; and (ii) administering a therapeutically effective amount of cannabis flower to a patient; wherein said patient experiences reduced symptoms due to said cannabis flower administration.

In some embodiments, the present invention teaches a method of treating Type 1 narcolepsy, said method comprising: (i) identifying a patient with Type 1 narcolepsy; and (ii) administering a therapeutically effective amount of cannabis extract to a patient; wherein said patient experiences reduced symptoms due to said cannabis extract administration.

In some embodiments, the present invention teaches a method of treating Type 1 narcolepsy, said method comprising: (i) identifying a patient with Type 1 narcolepsy; and (ii) administering a therapeutically effective amount of cannabis composition to a patient; wherein said patient experiences reduced symptoms due to said cannabis composition administration.

In some embodiments, the present invention teaches a method of treating Type 2 narcolepsy, said method comprising: (i) identifying a patient with Type 2 narcolepsy; and (ii) administering a therapeutically effective amount of cannabis flower to a patient; wherein said patient experiences reduced symptoms due to said cannabis flower administration.

In some embodiments, the present invention teaches a method of treating Type 2 narcolepsy, said method comprising: (i) identifying a patient with Type 2 narcolepsy; and (ii) administering a therapeutically effective amount of cannabis extract to a patient; wherein said patient experiences reduced symptoms due to said cannabis extract administration.

In some embodiments, the present invention teaches a method of treating Type 2 narcolepsy, said method comprising: (i) identifying a patient with Type 2 narcolepsy; and (ii) administering a therapeutically effective amount of cannabis composition to a patient; wherein said patient experiences reduced symptoms due to said cannabis composition administration.

In some embodiments, the present invention teaches a method of treating cataplexy, said method comprising: (i) identifying a patient with cataplexy; and (ii) administering a therapeutically effective amount of cannabis flower to a patient; wherein said patient experiences reduced symptoms due to said cannabis flower administration.

In some embodiments, the present invention teaches a method of treating cataplexy, said method comprising: (i) identifying a patient with cataplexy; and (ii) administering a therapeutically effective amount of cannabis extract to a patient; wherein said patient experiences reduced symptoms due to said cannabis extract administration.

In some embodiments, the present invention teaches a method of treating cataplexy, said method comprising: (i) identifying a patient with cataplexy; and (ii) administering a therapeutically effective amount of cannabis composition to a patient; wherein said patient experiences reduced symptoms due to said cannabis composition administration.

In some embodiments, the present invention teaches a method of treating sleep apnea, said method comprising: (i) identifying a patient with sleep apnea; and (ii) administering a therapeutically effective amount of cannabis flower to a patient; wherein said patient experiences reduced symptoms due to said cannabis flower administration.

In some embodiments, the present invention teaches a method of treating sleep apnea, said method comprising: (i) identifying a patient with sleep apnea; and (ii) administering a therapeutically effective amount of cannabis extract to a patient; wherein said patient experiences reduced symptoms due to said cannabis extract administration.

In some embodiments, the present invention teaches a method of treating sleep apnea, said method comprising: (i) identifying a patient with sleep apnea; and (ii) administering a therapeutically effective amount of cannabis composition to a patient; wherein said patient experiences reduced symptoms due to said cannabis composition administration.

In some embodiments, the present invention teaches a method of treating circadian rhythm disorders, said method comprising: (i) identifying a patient with circadian rhythm disorders; and (ii) administering a therapeutically effective amount of cannabis flower to a patient; wherein said patient experiences reduced symptoms due to said cannabis flower administration.

In some embodiments, the present invention teaches a method of treating circadian rhythm disorders, said method comprising: (i) identifying a patient with circadian rhythm disorders; and (ii) administering a therapeutically effective amount of cannabis extract to a patient; wherein said patient experiences reduced symptoms due to said cannabis extract administration.

In some embodiments, the present invention teaches a method of treating circadian rhythm disorders, said method comprising: (i) identifying a patient with circadian rhythm disorders; and (ii) administering a therapeutically effective amount of cannabis composition to a patient; wherein said patient experiences reduced symptoms due to said cannabis composition administration.

In some embodiments, the present invention teaches a method of treating restless leg syndrome, said method comprising: (i) identifying a patient with restless leg syndrome; and (ii) administering a therapeutically effective amount of cannabis flower to a patient; wherein said patient experiences reduced symptoms due to said cannabis flower administration.

In some embodiments, the present invention teaches a method of restless leg syndrome, said method comprising: (i) identifying a patient with restless leg syndrome; and (ii) administering a therapeutically effective amount of cannabis extract to a patient; wherein said patient experiences reduced symptoms due to said cannabis extract administration.

In some embodiments, the present invention teaches a method of treating restless leg syndrome, said method comprising: (i) identifying a patient with restless leg syndrome; and (ii) administering a therapeutically effective amount of cannabis composition to a patient; wherein said patient experiences reduced symptoms due to said cannabis composition administration.

In some embodiments, the present invention teaches a method of treating any sleep disorder, said method comprising: (i) identifying a patient with sleep disorder; and (ii) administering a prescribed amount of any cannabis composition described herein to a patient; wherein said patient experiences reduced symptoms due to said cannabis administration.

In some embodiments, the present application teaches a cannabis flower or variety having the pharmacological properties of the cannabis profile, wherein the flower or variety prophylactically or therapeutically treats one or more conditions selected from the group consisting of Type 1 narcolepsy, Type 2 narcolepsy, isolated cataplexy, sleep apnea, circadian rhythm disorders, and restless leg syndrome.

In some embodiments, the present application teaches a cannabis extract having the pharmacological properties of the cannabis profile, wherein the extract prophylactically or therapeutically treats one or more conditions selected from the group consisting of Type 1 narcolepsy, Type 2 narcolepsy, isolated cataplexy, sleep apnea, circadian rhythm disorders, and restless leg syndrome.

In some embodiments, the present application teaches a cannabis composition having the pharmacological properties of the cannabis profile, wherein the composition prophylactically or therapeutically treats one or more conditions selected from the group consisting of Type 1 narcolepsy, Type 2 narcolepsy, isolated cataplexy, sleep apnea, circadian rhythm disorders, and restless leg syndrome.

In some embodiments, the present application teaches a cannabis flower or variety, said flower or variety comprising a pharmacological properties of the cannabis profile prophylactically or therapeutically treats one or more conditions selected from the group consisting of Type 1 narcolepsy, Type 2 narcolepsy, isolated cataplexy, sleep apnea, circadian rhythm disorders, and restless leg syndrome based on the of the composition, and wherein the cannabis flower or variety comprises at least 1.5% terpene content by weight, wherein the terpene profile consists of camphene, carene, β-caryophyllene, caryophyllene oxide, fenchol, guaiol, α-humulene, limonene, linalool, myrcene, nerolidol, β-ocimene, α-phelladrene, phytol, α-pinene, β-pinene, pinoline, γ-terpinene, 60 -terpinene, α-terpineol, and terpinolene of the compositions, and wherein the terpene contents are calculated based on dry weight of the flower or variety.

In some embodiments, the present application teaches a cannabis extract, said extract comprising a pharmacological properties of the cannabis profile prophylactically or therapeutically treats one or more conditions selected from the group consisting of Type 1 narcolepsy, Type 2 narcolepsy, isolated cataplexy, sleep apnea, circadian rhythm disorders, and restless leg syndrome based on the of the composition, and wherein the cannabis extract comprises at least 1.5% terpene content by weight, wherein the terpene profile consists of camphene, carene, β-caryophyllene, caryophyllene oxide, fenchol, guaiol, α-humulene, limonene, linalool, myrcene, nerolidol, β-ocimene, α-phelladrene, phytol, α-pinene, β-pinene, pinoline, γ-terpinene, α- terpinene, α-terpineol, and terpinolene of the compositions, and wherein the terpene contents are calculated based on dry weight of the extract.

In some embodiments, the present application teaches a cannabis composition, said cannabis composition comprising: (i) at least one cannabis extract; (ii) at least one isolated cannabis compound; wherein the composition is tailored for a specific medicinal purpose to treat one or more conditions selected from the group consisting of Type 1 narcolepsy, Type 2 narcolepsy, isolated cataplexy, sleep apnea, circadian rhythm disorders, and restless leg syndrome based on the pharmacological properties of the cannabis profile of the composition, and wherein the cannabis composition comprises at least 1.5% terpene oil content, wherein the terpene profile consists of camphene, carene, β-caryophyllene, caryophyllene oxide, fenchol, guaiol, α-humulene, limonene, linalool, myrcene, nerolidol, β-ocimene, α-phelladrene, phytol, α-pinene, β-pinene, pinoline, γ-terpinene, α-terpinene, α-terpineol, and terpinolene of the compositions, wherein the terpene oil content is determined by the additive content of the terpenes in the terpene profile, and wherein the terpene contents are calculated based on dry weight of the composition.

In some embodiments, the methods of treating diseases of the present application utilize one or more cannabis plants, plant parts, tissues, or cells described herein.

In some embodiments, the methods of treating diseases of the present application utilize one or more cannabis extracts described herein.

In some embodiments, the methods of treating diseases of the present application utilize one or more cannabis compositions described herein.

In some embodiments, the methods of treating diseases of the present application administer cannabis edible products described herein.

In some embodiments, the methods of treating diseases of the present application administer cannabis tinctures for smoking or vaporization described herein.

In some embodiments, the methods of treating diseases of the present application administer cannabis transdermal delivery patches described herein.

### BRIEF DESCRIPTION OF THE FIGURES

FIG. 1 shows results of an HPLC analysis of raw cannabis flower used to treat narcolepsy and other sleep disorders, providing its cannabis profile, and also used to produce cannabis compositions in FIGS. 4, 5 and 6, providing its cannabis profile.

FIG. 2 shows results of an HPLC analysis of raw cannabis flower used to treat narcolepsy and other sleep disorders.

FIG. 3 shows results of an HPLC analysis of a cannabis composition used to treat narcolepsy and other sleep disorders, providing its cannabis profile.

FIG. 4 shows results of an HPLC analysis of another cannabis composition used to treat narcolepsy and other sleep disorders, providing its cannabis profile.

FIG. 5 shows results of an HPLC analysis of yet another cannabis composition used to treat narcolepsy and other sleep disorders, providing its cannabis profile.

FIG. 6 illustrates an example extraction process used to obtain a cannabis extract from cannabis flower or plant parts.

FIG. 7 is Plot of HPLC result from a high-THC sample.

FIG. 7 is plot of HPLC result from a high-THC CBD sample.

FIG. 8 is plot of HPLC result from a cannabis extract for an embodiment of this application.

FIG. 9 is plot of GC-MS for a terpene-rich sample.

FIG. 10 is is plot of HPLC result from a high-THC sample.

### DETAILED DESCRIPTION OF THE EMBODIMENTS

### Definitions

Before describing the embodiments, it should be noted that it is not limited to herein described methods and experimental conditions, as well as the terminology used herein for describing particular embodiments is not intended to be limiting. Unless defined otherwise, all technical and scientific terms used herein have the meaning as commonly understood by one of ordinary skill in the art to which this invention pertains. Although any methods and materials similar or equivalent to those described herein can be used in the practice or testing of the invention, particular methods and materials are now described.

As used herein, the following terms and phrases shall have the meanings set forth below. Unless defined otherwise, all technical and scientific terms used herein have the same meaning as commonly understood to one of ordinary skill in the art.

“Type 1 Narcolepsy” is narcolepsy with cataplexy. “Type 2 narcolepsy” is narcolepsy without cataplexy.

“Cataplexy” or “Isolated Cataplexy” is a sudden and uncontrollable muscle weakness or paralysis that comes on during the day and is often triggered by a strong emotion, such as excitement or laughter. A person experiencing total cataplexy stays awake and is aware of what is happening, but cannot move. These episodes last up to a minute or two, and some people may fall asleep afterwards.

“Sleep Apnea” sleep disorder in which breathing repeatedly stops and starts, it includes obstructive sleep apnea, central sleep apnea and complex sleep apnea syndrome.

“Circadian Rhythm Disorders” are a family of sleep disorders affecting (among other bodily processes) the timing of sleep. People with circadian rhythm sleep disorders are unable to go to sleep and awaken at the times commonly required for work and school as well as social needs. They are generally able to get enough sleep if allowed to sleep and wake at the times dictated by their “body clocks”. The quality of their sleep is usually normal unless they also have another sleep disorder. Disorders include shift work sleep disorder, advanced sleep phase disorder, delayed sleep phase disorder, irregular sleep-wake rhythm disorder, and non-24 hour sleep-wake disorder.

“Restless Leg Syndrome,” also known as Willis-Ekbom Disease, is a sleep disorder that is characterized by an overwhelming urge to move the legs when they are at rest. The urge to move the legs is usually, but not always, accompanied by unpleasant sensations. It is less common but possible to have symptoms in the arms, face, torso, and genital region. Symptoms occur during inactivity and they are temporarily relieved by movement or pressure. Symptoms are most severe in the evening and nighttime hours and can profoundly disrupt a patient's sleep and daily life.

Cannabis plants. As used herein, the term “plant” refers to plants in the genus of cannabis and plants derived thereof. Such as cannabis plants produced via asexual reproduction and via seed production.

As used herein, the term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”. Plant part may also include certain extracts such as kief or hash which includes cannabis trichomes or glands.

The term “raw cannabis flower” means unprocessed cannabis flower bud. Raw cannabis flower, is typically dried without heat for the purposes of being consumed in edible or smoked form.

The terms “cannabis variety,” “cannabis strain,” “variety,” or “strain” are used interchangeably herein and mean a type of specialty cannabis that share a uniform morphological or physiological character, or more specifically means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

The term “cannabis extract” is created by processing raw cannabis flower, cannabis plants, or other cannabis plant parts to extract and concentrate cannabis compounds. It should be noted that some, but not all, methods of processing of cannabis into a cannabis extract change the terpene and cannabinoid profile of the original cannabis, such that the cannabis extract has a slightly altered cannabis profile.

The term “cannabis profile” means the ratio by weight of cannabinoids and/or terpenes in cannabis plants, plant parts, extracts or compositions.

The term “cannabis compounds” shall mean cannabinoids, terpenes or a mixture thereof.

The terms “cannabinoids” and “terpenes” include all known and unknown isomers, stereoisomers, diastereomers, and enantiomers of each, including those that are synthetic manufactured or naturally occurring in cannabis plants or plant parts.

Compounds that have the same molecular formula but differ in the nature or sequence of bonding of their atoms or the arrangement of their atoms in space are termed “isomers.” Isomers that differ in the arrangement of their atoms in space are termed “stereoisomers.” “Diastereomers” are stereoisomers with opposite configuration at one or more chiral centers which are not enantiomers. Stereoisomers bearing one or more asymmetric centers that are non-superimposable mirror images of each other are termed “enantiomers.”

The term “cannabis” used alone, without a modified noun, means cannabis compounds, cannabis plants, cannabis plant parts, raw cannabis flower, cannabis extracts or cannabis compositions.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil, coconut oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A “substantial pure” composition, mixture or extract of cannabinoid is defined as a preparation having a chromatographic purity (of the desired cannabinoid) of greater than 90%, more preferably greater than 95%, more preferably greater than 96%, more preferably greater than 97%, more preferably greater than 98%, more preferably greater than 99% and most preferably greater than 99.5%, as determined by HPLC.

The term “substantially free” can be taken to mean that no cannabinoids other than the target cannabinoid are detectable by HPLC.

A “participant”, “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compounds and compositions, including but not limited to raw cannabis flower, cannabis plants, plant parts, cannabis extracts and cannabis compositions described herein. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating narcolepsy, cataplexy, other related diseases or any other medical condition, is well understood in the art, and includes ingestion or administration of a composition which reduces the frequency of, or delays the onset of, symptoms of a medical condition in a subject relative to a subject which does not receive the composition.

The phrase “therapeutically effective amount” is an art-recognized term. In certain embodiments, the term refers to an amount of cannabis disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

As used herein, the term “couch lock” is described as a heavy body high which reduces the ability of users to function, and is associated with lethargy and lack of motivation, but is better defined as time limited but profound psychomotor retardation limiting a subject's ability to function. In some embodiments, the cannabis described herein reduces the myrcene “couch lock” effects by changing the cannabis profile by reducing the myrcene levels or increasing levels of other cannabis compounds that counteract couch lock.

In certain embodiments, the cannabis described herein are formulated in a manner such that said cannabis will be delivered to a patient in a therapeutically effective amount, as part of a prophylactic or therapeutic treatment. The desired amount of the cannabis to be administered to a patient will depend on absorption, distribution, metabolic inactivation, and excretion rates of the cannabis, as well as the delivery rate of same. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular cannabis may be adjusted to accommodate variations in the treatment parameters. Such treatment parameters include the clinical use to which the preparation is put, e.g., the site treated, the type of patient, e.g., human or non-human, adult or child, and the nature of the disease or condition.

### Cannabis and Cannabinoids

Cannabis, more commonly known as marijuana, is a genus of flowering plants that includes at least three species, Cannabis sativa, Cannabis indica, and Cannabis ruderalis as determined by plant phenotypes and secondary metabolite profiles. Cannabis plants have existed and been cultivated for millennia, and more recently have become the target of prolific cross-breeding. Thus, cannabis actually encompasses a wide variety of plants that produce a breadth of compounds, each with potential for medical treatment of various diseases.

Although cannabis and the compounds, extracts and compositions derived therefrom can be used to treat several symptoms of narcolepsy, isolated cataplexy, sleep apnea, and related sleep disorders it is not a one size fits all cure. Diseases, including narcolepsy, sleep apnea, and related sleep disorders may be treated more effectively if cannabis medicines are used to treat the symptoms thereof are tailored to each disease with specific cannabis extracts and compositions.

TABLE 1
Non-limiting examples of naturally occurring cannabinoids

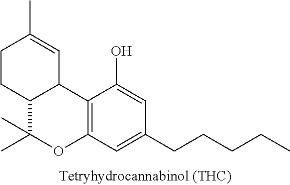

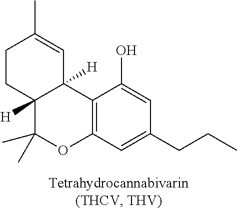 [SMILES: CCCc1cc(O)c2c(c1)OC(C)(C)[C@@H]1CCC(C)=C[C@@H]21]

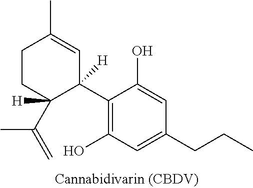 [SMILES: C=C(C)[C@@H]1CCC(C)=C[C@H]1c1c(O)cc(CCC)cc1O]

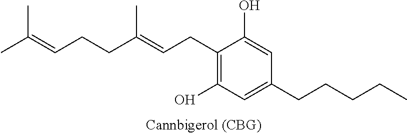 [SMILES: CCCCCc1cc(O)c(C/C=C(\C)CCC=C(C)C)c(O)c1]

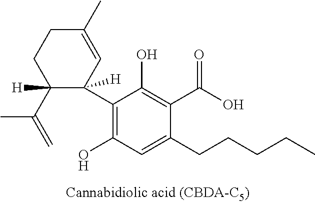 [SMILES: C=C(C)[C@@H]1CCC(C)=C[C@H]1c1c(O)cc(CCCCC)c(C(=O)O)c1O]

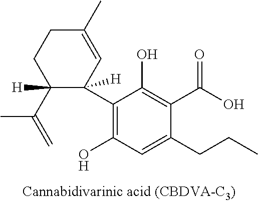 [SMILES: C=C(C)[C@@H]1CCC(C)=C[C@H]1c1c(O)cc(CCC)c(C(=O)O)c1O]

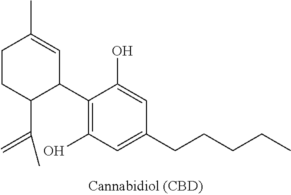 [SMILES: C=C(C)C1CCC(C)=CC1c1c(O)cc(CCCCC)cc1O]

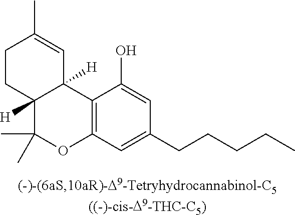 [SMILES: CCCCCc1cc(O)c2c(c1)OC(C)(C)[C@@H]1CCC(C)=C[C@@H]21]

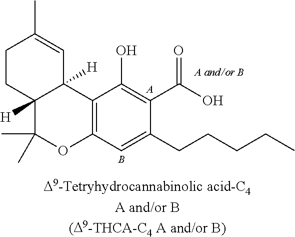

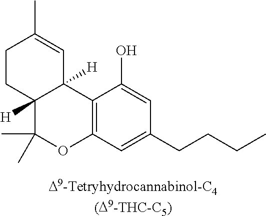 [SMILES: CCCCc1cc(O)c2c(c1)OC(C)(C)[C@@H]1CCC(C)=C[C@@H]21]

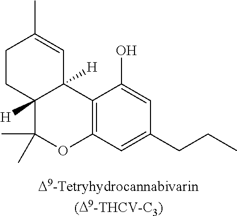 [SMILES: CCCc1cc(O)c2c(c1)OC(C)(C)[C@@H]1CCC(C)=C[C@@H]21]

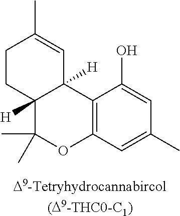

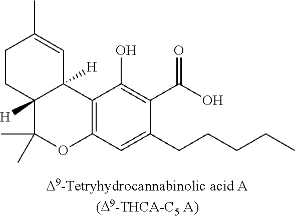 [SMILES: CCCCCc1cc2c(c(O)c1C(=O)O)[C@@H]1C=C(C)CC[C@H]1C(C)(C)O2]

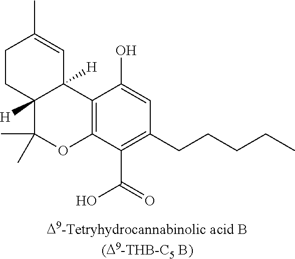 [SMILES: CCCCCc1cc(O)c2c(c1C(=O)O)OC(C)(C)[C@@H]1CCC(C)=C[C@@H]21]

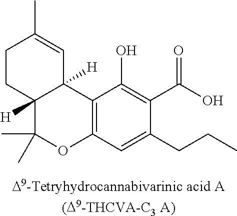 [SMILES: CCCc1cc2c(c(O)c1C(=O)O)[C@@H]1C=C(C)CC[C@H]1C(C)(C)O2]

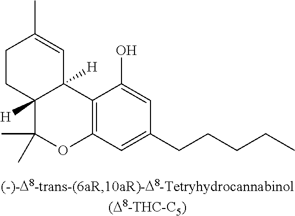 [SMILES: CCCCCc1cc(O)c2c(c1)OC(C)(C)[C@@H]1CCC(C)=C[C@@H]21]

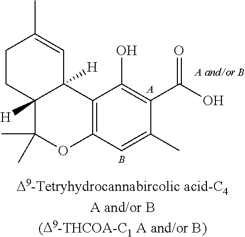

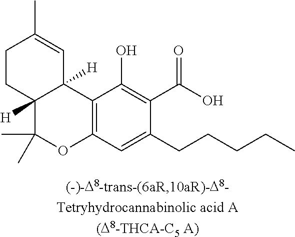 [SMILES: CCCCCc1cc2c(c(O)c1C(=O)O)[C@@H]1C=C(C)CC[C@H]1C(C)(C)O2]

Cannabinoids are a class of diverse chemical compounds that activate cannabinoid receptors in mammals. Cannabinoids produced by plants are called phytocannabinoids, a.k.a., natural cannabinoids, herbal cannabinoids, and classical cannabinoids. At least 85 different cannabinoids have been isolated from the cannabis plants (El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42; Brenneisen, supra). Typical cannabinoids isolated from cannabis plants include, but are not limited to, THC ( 4⁹-Tetrahydrocannabinol), CBD (Cannabidiol), CBG (Cannabigerol), CBC (Cannabichromene), CBL (Cannabicyclol), CBV (Cannabivarin), THCV (Δ⁹-Tetrahydrocannabivarin), CBDV (Cannabidivarin), CBCV (Cannabichromeovarin), CBGV (Cannabigerovarin), and CBGM (Cannabigerol Monomethyl Ether). These are discussed in more depth below.

In the more common cannabis strains, the principle cannabinoids present in cannabis are the cannabinoid acids Δ⁹-tetrahydrocannabinolic acid (THCA), and cannabidiolic acid (CBDA), with small amounts of the corresponding neutral cannabinoids, respectively Δ⁹ tetrahydrocannabinol (THC) and cannabidiol (CBD).

In addition to these principle cannabinoids, cannabis typically contains other minor cannabinoids including but not limited to those listed above, and some of which are described herein. Other cannabinoids may be intermediates in the biosynthesis of the major cannabinoids and hence exist at only low levels in the plant as they are constantly undergoing further biotransformation once they are formed. An example of such a cannabinoid is cannabigerol (CBG). Other minor cannabinoids may represent the end point of an alternative biosynthetic pathway to that leading to the formation of the major cannabinoids THC and CBD. These cannabinoids are frequently relatively more abundant in the plant, an example being cannabichromene (CBC).

A special example of a minor cannabinoid that is the end point of a biosynthetic pathway is Δ⁹-tetrahydrocannabivarinic acid (THCVA) which is a cannabinoic acid present in some cannabis plants and plant parts. Δ⁹-Tetrahydrocannabivarin (THCV) is the corresponding neutral cannabinoid, which is also present in in some strains. In most varieties, THCVA is more prevalent in raw cannabis than THCV, however both compounds may be found in raw cannabis of a number of different cannabis strains. Further, THCVA in raw cannabis will degrade to THCV over time. Similarly extracting, curing, other processing, or smoking of raw cannabis, typically degrades THCVA leaving THCV.

THCVA is a homologue of THCA. THCVA differs in structure from THCA due to the presence of a propyl (C₃H₇) side chain rather than a pentyl (C₅H₁₁) side chain on the aromatic ring. Analogously, THCV is a homologue of THC. THCV differs in structure from THC due to the presence of a propyl (C₃H₇) side chain rather than a pentyl (C₅H₁₁) side chain on the aromatic ring.

These compounds usually accompany THCA and/or THC at a level of 1-2% of THCA and/or THC present. However, in certain selectively bred varieties of cannabis THCV or THCVA can account for greater than 70% of total cannabinoids, with THCA and THC being reduced to the level of minor constituents. In other varieties, THCVA or THCV may have closer to a 1:1 ration with THCA and/or THC.

THCV is known to be a CB₁ and CB₂ receptor antagonist, and it blocks the effects of THC. The inventors have also recognized that THCV is an antagonist to the CB₁ and CB₂ receptor at low doses, but an agonist of the CB₁ and CB₂ receptor at higher doses. The CB₁ and CB₂ receptors are located throughout the body and especially the brain; the evidence that THCV is active at these receptor sites has been well documented (e.g., Stevenson et al, 2005). Many of the studies conducted to date involve the brain and both neurological and psychiatric disorders.

In neurology, evidence may show that THCV has anti-seizure and anti-epileptic effects in both human and animal models (Devinsky et al, 2014; Hill et al, 2012; Hill et al, 2010; Dennis et al, 2008). In other areas of neurology, some research shows THCV may treat Parkinson's (Garcia et al, 2011) and ALS. In narcolepsy, it is thought that the mechanism of action may be through the same CB₁ and CB₂ receptor sites and research into other sleep related disorders are ongoing (Calik & Carley, 2017).

The inventors have recognized that THCV and/or THCVA at equal or higher volume may compliment THC in one or more synergistic ways that may contribute to treatment efficacy for symptoms of narcolepsy, isolated cataplexy, sleep apnea, circadian rhythm disorders and/or restless leg syndrome.

The inventors have recognized that THCV and/or THCVA alone may treat symptoms of narcolepsy, isolated cataplexy, sleep apnea, circadian rhythm disorders and/or restless leg syndrome.

The inventors have also recognized through review of studies not directly relative to narcolepsy treatment that THCV counteracts or is an antagonist to the sedative properties often referred to as “couch lock” of the mono-terpene myrcene, which may be found in higher volume in strains with high THC content.

### Cannabis and Terpenes

In addition to cannabinoids, cannabis contains dozens of terpene compounds. Terpenes are a large and diverse class of organic compounds, produced by a variety of plants. They are often strong smelling and thus may have had a protective function. Terpenes are derived biosynthetically from units of isoprene, which has the molecular formula C₅H₈. Cannabis plants produce at over 120 different terpenes at different levels in their trichomes. Age, maturation and time of day can affect the amount and ratios of terpenes. Climate and weather also affect terpenes and flavonoid production.

In addition to many circulatory and muscular effects, some terpenes interact with neurological receptors. A few terpenes produced by cannabis plants also bind weakly to cannabinoid receptors. Some terpenes can alter the permeability of cell membranes and allow in either more or less cannabinoids, while other terpenes can affect serotonin and dopamine receptors as neurotransmitters.

Some common terpenes found in cannabis, at detectable levels, include but are not limited to, camphene, carene, β-caryophyllene, caryophyllene oxide, fenchol, guaiol, α-humulene, limonene, linalool, myrcene, nerolidol, β-ocimene, α-phelladrene, phytol, α-pinene, β-pinene, pinoline, γ-terpinene, α-terpinene, α-terpineol, and terpinolene.

Terpenes have been shown to have their own medicinal qualities as well as synergistic effects with cannabinoids. This is known as the entourage effect and is generally considered to result in plants providing advantages over only using the natural products that are isolated from them (Russo 2011, Taming THC: potential cannabis synergy and phytocannabinoid-terpenoid entourage effects, British Journal of Pharmacology, 163:1344-1364).

More recently it has been discovered that there are many more cannabinoids in cannabis that could be isolated and preserved in quantity. In addition, there are discovered terpenes such as linalool, myrcene, among others that are also now receiving focus in the industry as separate sources for additional desired medical efficacies. β-caryophyllene is often a predominant terpene found in cannabis. It is a selective full agonist at the CB₂ receptor, which makes it the only phytocannabinoid found outside the cannabis genus. In addition, it has anti-inflammatory and gastric cytoprotective properties, and may even have anti-malarial activity.

TABLE 2
A non-limiting list of the medical effects of some of the most common terpenes found.

| | Odor | | |
|---|---|---|---|
| Terpenoid | Description | Flavor Description | Suggested Pharmacology |
| α-pinene | Herbal, piney | Woody, piney, | Anti-inflammatory, |
| | | camphoraceous | bronchodilator, stimulant |
| camphene | Woody, piney | Camphoraceous, | Reduces plasma cholesterol |
| | | cooling, minty | and triglycerides, Antioxidant |
| | | | and free radical scavenger |
| β-pinene | Herbal, cooling, | Fresh, piney, woody | Strong antimicrobial |
| | piney | | |
| myrcene | Spicy, | Woody, vegetative, | Anti-inflammatory, sedative, |
| | herbaceous | citrus | antibiotic, analgesic |
| α-phellandrene | Terpenic, citrus | Terpenic, citrus, lime | Antinociceptive |
| carene | Citrus, sweet | None given | CNS depressant, anti- |
| | | | inflammatory |
| α-terpinene | Woody, citrus, | Terpenic, woody, | Antioxidant |
| | medicinal | piney | |
| limonene | Citrus, fresh | Sweet, orange, citrus | Anxiolytic, antidepressant, |
| | | | immunostimulant |
| β-ocimene | Floral, green | Green, tropical, woody | Possible anti-bacterial |
| γ-terpinene | Terpenic, woody | Terpenic, citrus, lime- | Antioxidant |
| | | like | |
| terpinolene | Herbal, woody | Sweet, fresh, piney, | Comforting, calming, anti- |
| | | citrus | oxidant, antifungal |
| linalool | Floral, citrus | Citrus, orange, lemon, | Sedative, anxiolytic, |
| | | floral | immunostimulant |
| fenchol | Camphor, piney | Fresh, piney | Possible stimulant |
| α-terpineol | Floral, piney | None given | Sedative, AChE inhibitor, |
| | | | antioxidant |
| β-caryophyllene | Spicy, woody | Spicy, clove, rosemary | Selective agonist of CB₂ |
| | | | receptor, anti-inflammatory, |
| | | | antimalarial |
| α-humulene | Woody | None given | Anti-inflammatory |
| caryophyllene | Woody, sweet | None given | Antifungal, stimulant |
| oxide | | | |

Cannabis plants, extracts and compositions.

Cannabis cultivation techniques including cloning to preserve ratios of cannabinoids and seed stabilization and cross breeding has produced specific cannabis varieties that may be regarded as selectively therapeutic for certain medical conditions. A focus of this application is in component selectivity when creating or selecting cannabis varieties, extracts and compositions that have THCV and/or THCVA as a primary cannabinoid.

Thus, in some embodiments cannabis plants, plant parts, plant tissues and plant cells will have a THC content selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70% , 71%, 72%, 73%, 74%, 75%, 76%, 77% 78%, 79%, 80%, 81%, 82%, 83%, 84%, 85%, 86%, 87%, 88%, 89% and 90%; and, a THCV content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 94%, 95%, 96%, 97%, 98%, 98.5%, 99%, 99.5%, 99.8%, 99.9% and 100%.

Thus, in some embodiments cannabis plants, plant parts, plant tissues and plant cells will have a TWA content selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32% 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89% and 90%; and, a THCVA content elected from the group consisting of 1.5%, 1.6%, 1.7%, 1.8%, 1.9%, 10%, 11.%, 2.0%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 94%, 95%, 96%, 97%, 98%, 98.5%, 99%, 99.5%, 99.8%, 99.9% and 100%.

Thus, in some embodiments cannabis plants, plant parts, plant tissues and plant cells will have content comprising THC and/or THCA selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, content comprising THVC and/or THCVA content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0% 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 94%, 95%, 96%, 97%, 98%, 98.5%, 99%, 99.5%, 99.8%, 99.9% and 100%.

Thus, in some embodiments raw cannabis flower will have a THC content selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%. 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, a THCV content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26,0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0% 92%, 93%, 94%, 95%, 96%, 97%, 98%, 98.5%, 99%, 99.5%, 99.8%, 99.9% and 100%.

Thus, in some embodiments raw cannabis flower will have a THCA content selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%. 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, a THCVA content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%) 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 94%, 95%, 96%, 97%, 98%, 98.5%, 99%, 99.5%, 99.8%, 99.9%, and 100%.

Thus, in some embodiments raw cannabis flower will have content comprising THC and/or THCA selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90% and, content comprising THVC and/or THCVA content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8.%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67,0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 940, 95%, 96% 97%, 98%, 98.5%, 99%, 99.5%, 99.8%, 99.9% and 100%.

Thus, in some embodiments cannabis extracts will have a THC content selected from the group consisting of 0%, 0.1%, 0.2%, 0.5%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 87%, 88%, 89%, and 90%); and, a THCV content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38,0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 67.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 94%, 95%, 96%, 97%, 98%, 98.5%, 99%, 99.5%, 99.8%, 99.9% and 100%.

Thus, in some embodiments cannabis extracts will have a THCA content selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56% 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, a THCVA content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27,0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 8930%, 90.0%, 91.0%, 92%, 93%, 94%, 95%, 96%, 97%, 98%, 98.5%, 99%, 99.5%, 99.8%, 99.9% and 100%.

Thus, in some embodiments cannabis extracts will have content comprising THC and/or THCA selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, content comprising THVC and/or THCVA content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.O%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89,0%, 90.0%, 91.0%, 92%, 93%, 94%, 95%, 96%, 97%, 98%, 98.5%, 99%, 99.5%, 99.8%, 99.9% and 100%.

Thus, in some embodiments cannabis compositions will have a THC content selected from the group consisting of 0%, 0.1%. 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, a THCV content selected from the group consisting of 1.5%, 1.6%, 1.7%, 1.8%, 1.9%, 2.0%, 2.1%, 2.2%, 2.3%, 2.4%, 2.5, 2.6%, 2.7%, 2.8%, 2.9%, 3.0%, 4.0%, 5.0%. 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24,0%, 25.0%, 26.0%, 27.0%, 28.0%, 29.0%, 30.0%, 31.0%, 32.0%, 33.0%, 34.0%, 35.0%, 36.0%, 37.0%, 38,0%, 39.0%, 40.0%, 41.0%, 42,0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93% 94%, 95%, 96%, 97%, 98%, 98.5%, 99%, 99.5%, 99.8%, 99.9% and 100%.

Thus, in some embodiments cannabis compositions will have a THCA content selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, a THCVA content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29,0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92%, 93%, 94%, 95%, 96%, 97%, 98%, 98.5%, 99%, 99.5%, 99.8%, 99.9% and 100%.

Thus, in some embodiments cannabis compositions will have content comprising THC and/or THCA selected from the group consisting of 0%, 0.1%, 0.2%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% and 90%; and, content comprising THVC and/or THCVA content selected from the group consisting of 1.5%, 1.6%, 1.7%, 1.8%, 1.9%, 2.0%, 2.1%, 2.2%, 2.3%, 2.4%, 2.5%, 6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63,0%, 64.0%, 65.0%, 66.0%, 67,0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89,0%, 90.0%, 91.0%, 92%, 93%, 94%, 95%, 96%, 97%, 98%, 98.5%, 99%, 99.5%, 99.8%, 99.9% and 100%.

In some embodiments, the cannabis composition of the present invention comprises at least 0.05% content by weight of at least two terpenes of said terpene profile.

In some embodiments, the cannabis composition of the present invention comprises at least 0.05% content by weight of at least three, four, five, six, seven, eight, or nine terpenes of said terpene profile.

In some embodiments, cannabis compositions described herein comprise at least 2% content by weight of at least two cannabinoids selected from the group consisting of: THC, CBD, CBG, CBC, THCV, CBDV, and cannabigevarin (CBGV).

In some embodiments, the cannabis composition of the present invention comprises at least 2% content by weight of at least three, four, or five cannabinoids selected from the group consisting of: THC, CBD, CBG, CBC, THCV, CBDV, CBGV.

In some embodiments, the cannabis composition may further comprise a terpene profile in which the first or second most abundant terpene in the terpene profile is α-pinene.

In some embodiments, the cannabis composition may further comprise a terpene profile in which the first or second most abundant terpene in the terpene profile is camphene.

In some embodiments, the cannabis composition may further comprise a terpene profile in which the first or second most abundant terpene in the terpene profile is β-pinene.

In some embodiments, the cannabis composition may further comprise a terpene profile in which the first or second most abundant terpene in the terpene profile is myrcene.

In some embodiments, the cannabis composition may further comprise a terpene profile in which the first or second most abundant terpene in the terpene profile is α-phellandrene.

In some embodiments, the cannabis composition may further comprise a terpene profile in which the first or second most abundant terpene in the terpene profile is limonene.

In some embodiments, the cannabis composition may further comprise a terpene profile in which the first or second most abundant terpene in the terpene profile is β-ocimene,

In some embodiments, the cannabis composition may further comprise a terpene profile in which the first or second most abundant terpene in the terpene profile is γ-terpinene,

In some embodiments, the cannabis composition may further comprise a terpene profile in which the first or second most abundant terpene in the terpene profile is terpinolene.

In some embodiments, the cannabis composition may further comprise a terpene profile in which the first or second most abundant terpene in the terpene profile is terpinolene.

In some embodiments, the cannabis composition may further comprise a terpene profile in which the first or second most abundant terpene in the terpene profile is linalool.

In some embodiments, the cannabis composition may further comprise a terpene profile in which the first or second most abundant terpene in the terpene profile is fenchol.

In some embodiments, the cannabis composition may further comprise a terpene profile in which the first or second most abundant terpene in the terpene profile is α-terpineol.

In some embodiments, the cannabis composition may further comprise a terpene profile in which the first or second most abundant terpene in the terpene profile is 13-caryophyllene.

In some embodiments, the THC:THCV ratio of the cannabis composition or the cannabis extract will be greater than or equal to 20:1, or 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In some embodiments, the THCA:THCVA ratio of the cannabis composition or the cannabis extract will be greater than or equal to 20:1, or 19:1, 18:1, 17:1, 16:1, 15:1, 14:1, 13:1, 12:1, 11:1 10:1, 9:1, 8:1, 7:1, 6:1, 5:1, 4:1, 3:1 2:1, 1:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In some embodiments the THCA and/or THC:THCVA and/or THCV ratio of the cannabis composition or the cannabis extract will be greater than or equal to 20:1, or 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In some embodiments, the THC:THCV ratio of raw cannabis flower will be greater than or equal to 20:1, or 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In some embodiments, the THCA:THCVA ratio of raw cannabis flower will be greater than or equal to 20:1, or 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In some embodiments, the THCA and/or THC:THCVA and/or THCV ratio of raw cannabis flower will be greater than or equal to 20:1, or 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In any embodiment described herein, the THCA:THC ratio of a mixture used in a cannabis composition or the cannabis extract will be greater than or equal to 20:1, or 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3: 1:4, 1:5, 1:6, 1:7, 1:8, 1:9, 1:10, 1:11, 1:12: 1:13, 1:14, 1:15, 1:16, 1:17, 1:18, 1:19, 1:20, or lower.

In any embodiment described herein, the THCVA:THCV ratio of a mixture used in a cannabis composition or the cannabis extract will be greater than or equal to 20:1, or 19:1, 18:1, 17:1, 16:1, 15:1, 14.1, 13:1, 12:1, 11:1, 10:1, 9:1, 8:1, 7:1, 6:1, 5:1, 4:1, 3:1, 2:1, 1:1, 1:2, 1:3: 1:4, 1:5, 1:6, 1:7, 1:8; 1:9, 1:10, 1:11, 1:12: 1:13, 1:14, 1:15, 1:16, 1:17, 1:18, 1:19, 1:20, or lower.

In some embodiments, the cannabis composition may further comprise a terpene profile in which the first or second most abundant terpene in the terpene profile is α-humulene.

In some embodiments, the cannabis composition may further comprise a terpene profile in which the first or second most abundant terpene in the terpene profile is caryophyllene oxide.

In a preferred embodiment, the cannabis composition of the subject application is a cannabis extract or cannabis composition suitable for use in a vape pen cartridge.

In one embodiment, the cannabis composition of the subject application is a wax or paste that may be smoked, vaped, used in edibles, pills and other ingestible forms that may be administered to a patient.

In one embodiment, the cannabis extract is an oil used alone or mixed with one or more essential oils, creating a cannabis composition.

In one aspect, the cannabis extract is derived from blending or mixing of two or more other cannabis compounds.

A goal of the present invention is to preserve selected cannabis compounds, particularly THC, THCA, THCV, THCVA or some combination thereof, in a final cannabis composition or cannabis extract; wherein said composition or extract is an oil; and wherein the oil is dispensed via vapor cartridge or vape pen.

A goal of the present invention is to preserve selected cannabis compounds, particularly THC, THCA, THCV, THCVA or some combination thereof, in a final cannabis composition or cannabis extract; wherein said composition or extract is an oil; and wherein the oil is dispensed via vapor cartridge or vape pen; and wherein a patient may inhale or ingest the oil such that it has efficacy in treating at least one symptoms of at least one condition selected from the group consisting of narcolepsy, isolated cataplexy, sleep apnea, circadian rhythm sleep disorders and restless leg syndrome.

In some embodiments the vapor cartridge or vape pen carries an identification as to strain or strain mix and a cannabis profile having at least one cannabinoid listed on the cannabis profile.

It is one object of the present invention to preserve THC and THCV expressed in two or more strains of cannabis plant parts, whereby each strain is processed separately, and wherein the final cannabis compounds extracted from each strain are mixed together to form a single cannabis composition having approximately, equal amounts of THC and THCV, the cannabis compounds exceeding 50 percent of the weight of the cannabis composition up to being 100 percent of the cannabis composition.

It is one object of the present invention to preserve THCA and THCVA expressed in two or more strains of cannabis plant parts, whereby each strain is processed separately, and wherein the final cannabis compounds extracted from each strain are mixed together to form a single cannabis composition having approximately, equal amounts of THCA and THCVA, the cannabis compounds exceeding 50 percent of the weight of the cannabis composition up to being 100 percent of the cannabis composition.

One with skill in the art of cannabis processing and medicinal use thereof will appreciate that different cannabinoids produce different effects in general, and that terpenes also provide specific and separate effects in general. Synergy between THC and THCV cannabinoids in cannabis compositions and raw cannabis promote alert wakefulness in narcoleptic patients and/or patients with other sleep disorders described herein, even when said cannabis compositions or raw cannabis further comprise myrcene as the dominant terpene. Such effects on patients may be without side effect.

One with skill in the art of cannabis processing and medicinal use thereof will appreciate that different cannabinoids produce different effects in general, and that terpenes also provide specific and separate effects in general. Synergy between THCA and THCVA cannabinoids in cannabis compositions and raw cannabis promote alert wakefulness in narcoleptic patients and/or patients with other sleep disorders described herein, even when said cannabis compositions or raw cannabis further comprise myrcene as the dominant terpene. Such effects on patients may be without side effect.

One with skill in the art of cannabis processing and medicinal use thereof will appreciate that different cannabinoids produce different effects in general, and that terpenes also provide specific and separate effects in general. Synergy between THC, THCA, THCV and THCVA cannabinoids in cannabis compositions and raw cannabis promote alert wakefulness in narcoleptic patients and/or patients with other sleep disorders described herein, even when said cannabis compositions or raw cannabis further comprise myrcene as the dominant terpene. Such effects on patients may be without side effect.

One with skill in the art of cannabis processing and medicinal use thereof will appreciate that different cannabinoids produce different effects in general, and that terpenes also provide specific and separate effects in general. Synergy between THC and THCV cannabinoids and β-caryophyllene in cannabis compositions and raw cannabis promote alert wakefulness in narcoleptic patients and/or patients with other sleep disorders described herein. Such effects on patients may be without side effect, and may result in synergistic effects β-caryophyllene activate CB₁ and/or CB₂ receptors in patients.

One with skill in the art of cannabis processing and medicinal use thereof will appreciate that different cannabinoids produce different effects in general, and that terpenes also provide specific and separate effects in general. Synergy between THCA and THCVA cannabinoids and β-caryophyllene in cannabis compositions and raw cannabis promote alert wakefulness in narcoleptic patients and/or patients with other sleep disorders described herein. Such effects on patients may be without side effect, and may result in synergistic effects β-caryophyllene activate CB₁ and/or CB₂ receptors in patients.

One with skill in the art of cannabis processing and medicinal use thereof will appreciate that different cannabinoids produce different effects in general, and that terpenes also provide specific and separate effects in general. Synergy between THC, THCA, THCV and THCVA cannabinoids and β-caryophyllene in cannabis compositions and raw cannabis promote alert wakefulness in narcoleptic patients and/or patients with other sleep disorders described herein. Such effects on patients may be without side effect, and may result in synergistic effects β-caryophyllene activate CB₁ and/or CB₂ receptors in patients.

In one embodiment, the final cannabis composition is a mix of at least two separate cannabis varieties, each variety containing THCA and/or THCVA. In a preferred embodiment, a nominal 50% blend of THC and THCV may be determined most beneficial for general symptom treatment. The percentages just cited may be calculated from the total weight of the cannabinoids present in the composition.

In one embodiment, the final cannabis composition is a mix of at least two separate cannabis varieties, each variety containing THCA and/or THCVA. In a preferred embodiment, a nominal 50% blend of THCA and THCVA may be determined most beneficial for general symptom treatment. The percentages just cited may be calculated from the total weight of the cannabinoids present in the composition.

In one embodiment, the final cannabis composition is a mix of at least two separate cannabis varieties, each variety containing mixture of THC and THCA, and mixture of THCV, and THCVA. In a preferred embodiment, a nominal 50% blend of THC/THCA and THCV/THCVA may be determined most beneficial for general symptom treatment. The percentages just cited may be calculated from the total weight of the cannabinoids present in the composition.

In one embodiment, this application teaches a cannabis composition, wherein the cannabis composition is a mix of at least two separate cannabis varieties, each variety containing THC and/or THCV. In a preferred embodiment, a nominal 50% blend of THC and THCV may be determined beneficial in treating at least one symptoms of at least one condition selected from the group consisting of narcolepsy, isolated cataplexy, sleep apnea, circadian rhythm sleep disorders and restless leg syndrome. The percentages just cited may be calculated from the total weight of the cannabinoids present in the composition.

In one embodiment, this application teaches a cannabis composition, wherein the cannabis composition is a mix of at least two separate cannabis varieties, each variety containing THCA and/or THCVA. In a preferred embodiment, a nominal 50% blend of THCA and THCVA may be determined beneficial in treating at least one symptoms of at least one condition selected from the group consisting of narcolepsy, isolated cataplexy, sleep apnea, circadian rhythm sleep disorders and restless leg syndrome. The percentages just cited may be calculated from the total weight of the cannabinoids present in the composition

In one embodiment, this application teaches a cannabis composition, wherein the cannabis composition is a mix of at least two separate cannabis varieties, each variety containing mixture of THC and THCA, and mixture of THCV, and THCVA. In a preferred embodiment, a nominal 50% blend of THC/THCA and THCV/THCVA may be determined beneficial in treating at least one symptom of at least one condition selected from the group consisting of narcolepsy, isolated cataplexy, sleep apnea, circadian rhythm sleep disorders and restless leg syndrome. The percentages just cited may be calculated from the total weight of the cannabinoids present in the composition.

In one embodiment, this application teaches a therapeutically effective amount of a cannabis composition for treating one or more condition selected from the group consisting of narcolepsy, isolated cataplexy, and related disorders known and unknown, including but not limited to sleep apnea, circadian rhythm sleep disorders and restless leg syndrome, or any symptoms thereof, the composition comprising at least 50% by weight, of THC and THCV, calculated from the total weight of the cannabinoids present in the composition, and wherein the THC:THCV ratio by weight is 80:20 to 25:75, preferably 3:1 to 1:2, and in particular a range between 3.5:2.5 or 2.5:3.5.

In one embodiment, this application teaches a therapeutically effective amount of a cannabis composition for treating one or more condition selected from the group consisting of narcolepsy, isolated cataplexy, and related disorders known and unknown, including but not limited to sleep apnea, circadian rhythm sleep disorders and restless leg syndrome, or any symptoms thereof, the composition comprising at least 50% by weight, of THCA and THCVA calculated from the total weight of the cannabinoids present in the composition, and wherein the THCA:THCVA ratio by weight is 80:20 to 25:75, preferably 3:1 to 1:2, and in particular a range between 3.5:2.5 or 2.5:3.5.

In one embodiment, this application teaches a therapeutically effective amount of a cannabis composition for treating one or more condition selected from the group consisting of narcolepsy, isolated cataplexy, and related disorders known and unknown, including but not limited to sleep apnea, circadian rhythm sleep disorders and restless leg syndrome, or any symptoms thereof, the composition comprising at least 50% by weight, of THC/THCA and THCV/THCVA calculated from the total weight of the cannabinoids present in the composition, and wherein the THC/THCA:THCV/THCVA ratio by weight is 80:20 to 25:75, preferably 3:1 to 1:2, and in particular a range between 3.5:2.5 or 2.5:3.5.

In some embodiments, the present invention provides a composition may comprise at least one cannabinoid and at least one terpene for use in a method of treating, alleviating or reducing at least one symptom of a condition selected from the group consisting of narcolepsy, isolated cataplexy, sleep apnea, circadian rhythm sleep disorders and restless leg syndrome, wherein (a) said composition is derived from at least one of a cannabis plant enriched in THCV, a cannabis plant enriched in THC, a cannabis plant wherein the amounts of THC and THCV are substantially equal, (b) wherein at least one cannabinoid is selected from THC, and THCV, and (c) wherein said at least one terpene is selected from a group consisting of camphene, carene, β-caryophyllene, caryophyllene oxide, fenchol, gaiol, α-humulene, limonene, linalool, myrcene, nerolidol, β-ocimene, α-phelladrene, phytol, α-pinene, β-pinene, pinoline, γ-terpinene, α-terpinene, α-terpineol, and terpinolene.

In some embodiments, the present invention provides a composition may comprise at least one cannabinoid and at least one terpene for use in a method of treating, alleviating or reducing at least one symptom of a condition selected from the group consisting of narcolepsy, isolated cataplexy, sleep apnea, circadian rhythm sleep disorders and restless leg syndrome, wherein (a) said composition is derived from at least one of a cannabis plant enriched in

THCVA, a cannabis plant enriched in THC, a cannabis plant wherein the amounts of THCA and THCVA are substantially equal, (b) wherein at least one cannabinoid is selected from THCA, and THCVA, and (c) wherein said at least one terpene is selected from a group consisting of camphene, carene, β-caryophyllene, caryophyllene oxide, fenchol, gaiol, α-humulene, limonene, linalool, myrcene, nerolidol, β-ocimene, α-phelladrene, phytol, α-pinene, β-pinene, pinoline, γ-terpinene, α-terpinene, α-terpineol, and terpinolene.

In some embodiments, the present invention provides a composition may comprise at least one cannabinoid and at least one terpene for use in a method of treating, alleviating or reducing at least one symptom of at least one symptom of a condition selected from the group consisting of narcolepsy, isolated cataplexy, sleep apnea, circadian rhythm sleep disorders and restless leg syndrome, wherein (a) said composition is derived from at least one of a cannabis plant enriched in mixture of THCV/THCVA, a cannabis plant enriched in THC, a cannabis plant wherein the amounts of THC/THCA and THCV/THCVA are substantially equal, (b) wherein at least one cannabinoid is selected from THC/THCA, and THCV/THCVA, and (c) wherein said at least one terpene is selected from a group consisting of camphene, carene, β-caryophyllene, caryophyllene oxide, fenchol, gaiol, α-humulene, limonene, linalool, myrcene, nerolidol, β-ocimene, α-phelladrene, phytol, α-pinene, β-pinene, pinoline, γ-terpinene, α-terpinene, α-terpineol, and terpinolene.

In one embodiment of this invention the cannabis compositions or cannabis plants of the present invention are tailored to prevent, treat or ameliorate symptoms of at least one symptom of a condition selected from the group consisting of narcolepsy, isolated cataplexy, sleep apnea, circadian rhythm sleep disorders and restless leg syndrome. Effectiveness of the treatment will be confirmed by conducting a trial using double blind, randomized treatments comparing the effects of cannabis compositions containing THC and/or THCV, or combinations of other cannabinoid variants, and/or a combination of various terpenes. Concentrations used may be (0 mg, 0.5 mg, 1 mg, 2 mg, 5 mg, 10 mg, 15 mg, 20 mg or more THC) and/or (0.5 mg, 1 mg, 2 mg, 5 mg, 10 mg, 15 mg, 20 mg or more THCV) alone, or in combination with terpenes such as β-caryophyllene, myrcene, limonene, pinene, and/or linalool fortifiers. Terpene combinations will be chosen based on both their therapeutic activity (e.g. anti-convulsant properties of linalool) as well as flavor and organoleptic feel (e.g. cineole/eucalyptol for spicy flavor and cooling feel). The treatments may be administered 1 to 10 times per day.

In one embodiment of this invention the cannabis compositions or cannabis plants of the present invention are tailored to prevent, treat or ameliorate symptoms of at least one symptom of a condition selected from the group consisting of narcolepsy, isolated cataplexy, sleep apnea, circadian rhythm sleep disorders and restless leg syndrome. Effectiveness of the treatment will be confirmed by conducting a trial using double blind, randomized treatments comparing the effects of cannabis compositions containing THC and/or THCVA, or combinations of other cannabinoid variants, and/or a combination of various terpenes. Concentrations used may be approximately (0 mg, 0.5 mg, 1 mg, 2 mg, 5 mg, 10 mg, 15 mg, 20 mg or more THCA) and/or (0.5 mg, 1 mg, 2 mg, 5 mg, 10 mg, 15 mg, 20 mg or more THCVA) alone, or in combination with terpenes such as β-caryophyllene, myrcene, limonene, pinene, and/or linalool fortifiers. Terpene combinations will be chosen based on both of their therapeutic activity (e.g. anti-convulsant properties of linalool) as well as flavor and organoleptic feel (e.g. cineole/eucalyptol for spicy flavor and cooling feel). The treatments may be administered 1 to 10 times per day.

In one embodiment of this invention the cannabis compositions or cannabis plants of the present invention are tailored to prevent, treat or ameliorate symptoms of at least one symptom of a condition selected from the group consisting of narcolepsy, isolated cataplexy, sleep apnea, circadian rhythm sleep disorders and restless leg syndrome. Effectiveness of the treatment will be confirmed by conducting a trial using double blind, randomized treatments comparing the effects of cannabis compositions containing THC, THCA, THCV and/or THCVA, or combinations of other cannabinoid variants, and/or a combination of various terpenes. Concentrations used may be approximately (0 mg, 0.5 mg, 1 mg, 2 mg, 5 mg, 10 mg, 15 mg, 20 mg or more THC and/or THCA) and/or (0.5 mg, 1 mg, 5 mg, 10 mg, 15 mg, 20 mg or more THC and/or THCVA) alone, or in combination with terpenes such as β-caryophyllene, myrcene, limonene, pinene, and/or linalool fortifiers. Terpene combinations will be chosen based on both their therapeutic activity (e.g. anti-convulsant properties of linalool) as well as flavor and organoleptic feel (e.g. cineole/eucalyptol for spicy flavor and cooling feel). The treatments may be administered 1 to 10 times per day.

One object of the invention is to balance the level of THC and THCV preserved in an organic cannabis-based raw material or materials by way of processing the one or more raw materials to extract an oil or oils, the oil or oils in therapeutically effective amounts, including dose recommendation in treating with efficacy at least one symptom of a condition selected from the group consisting of narcolepsy, isolated cataplexy, sleep apnea, circadian rhythm sleep disorders and restless leg syndrome.

In some embodiments, inventors provide at least one method for treating a condition selected from the group consisting of narcolepsy, isolated cataplexy, sleep apnea, circadian rhythm sleep disorders and restless leg syndrome with a single cannabinoid compound, cannabis plant parts, cannabis extracts, cannabis compositions, or combination thereof. In some embodiments, the method may also be shown to be void of side effects associated with currently known pharmacological treatments.

In some embodiments, inventors provide at least one method for treating a condition selected from the group consisting of narcolepsy, isolated cataplexy, sleep apnea, circadian rhythm sleep disorders and restless leg syndrome with raw cannabis flower, one or more cannabis extract, or one or more cannabis compositions described in this application. In some embodiments, the method may also be shown to be void of side effects associated with currently known pharmacological treatments.

In some embodiments, inventors provide a method of treatment for narcoleptic patients by administering a therapeutically effective amount of raw cannabis flower, a cannabis extract, a cannabis composition or cannabis compound, any of which comprise tetrahydrocannabinol (THC) from 25 percent weight by volume to 75 percent weight by volume, the compound also including tetrahydrocannabivarin (THCV) from 25 percent weight by volume to 75 percent weight by volume.

In some embodiments, inventors provide a method of treatment for narcoleptic patients by administering a therapeutically effective amount of raw cannabis flower, a cannabis extract, a cannabis composition or cannabis compound, any of which comprise tetrahydrocannabinol (THCA) from 25 percent weight by volume to 75 percent weight by volume, the compound also including tetrahydrocannabivarin (THCVA) from 25 percent weight by volume to 75 percent weight by volume.

In some embodiments, inventors provide a method of treatment for narcoleptic patients by administering a therapeutically effective amount of raw cannabis flower, a cannabis extract, a cannabis composition or cannabis compound, any of which comprise tetrahydrocannabinol (THC/THCA) from 25 percent weight by volume to 75 percent weight by volume, the compound also including tetrahydrocannabivarin (THCV/THCVA) from 25 percent weight by volume to 75 percent weight by volume.

In some embodiments, a therapeutically effective amount of cannabis will be administered to patients via oral or inhaled routes.

In one embodiment, a therapeutically effective amount of cannabis will be administered to patients by taking pills over a period of time, the pills containing an oil, wax, or paste representing a form of the organic compound.

In one embodiment, a therapeutically effective amount of cannabis is administered by inhalation by vaping a cannabis extract or cannabis composition of the subject application during waking hours.

In one embodiment, a therapeutically effective amount of cannabis is inhalation by smoking raw cannabis flower, cannabis extract or cannabis composition of the subject application during waking hours.

In one embodiment of the invention, the inventors provide concentrated oil for inclusion into a vaporizing tool referred to as a vape pen in the industry. In one embodiment an amount of the oil having a nominally equal ratio of THC to THCV may be injected into a battery powered cartridge, which provides the heat to vaporize the product. In one example, a vape pen cartridge contains 0.5 g of cannabis composition, approx. 26% is THCV, approx. 35% is THC. For a narcoleptic patient, treatment is during the day time to maintain a wakeful feeling. Assuming 0.5 mg of vaped compound per single intake by a patient, it may be recommended to use a vape pen 1 to three times per hour taking one to 3 inhalations from the pen. In the case of the exact percentages mentioned above relative to ratio of THCV to THC, then each draw may include 1.3 mg THC and 1.75 mg THC.

Alternatively, a patient may smoke a “marijuana cigarette”, consume an edible or a drink containing the compound. In one aspect the cannabis composition is an oil blended with a skin penetration oil such as neem seed oil and a blood stream transdermal absorption agent, or for topical use. In yet another aspect the formula is regulated to a patch, such as in U.S. Pat. No. 9,655,634. In still another embodiment the formula is regulated to a pill form. Because of the unpredictable nature of some narcoleptic and cataplexic symptoms, it may be recommended that continual maintenance of the regimen chosen to dispense the described compounds having high THC and THCV ratio to approximately 1:1 at nominally 50% weights by volume be practiced during wake hours up until just before sleep time. In preferential circumstance, sufficient amounts of the prescribed compound remain in the patient's system during sleep hours for aiding symptoms of cataplexy such as sleep paralysis and narcoleptic hypnogogic and hypnopompic hallucinations.

In some embodiments, compositions described herein are derived from a female cannabis plants in a dosage form of an oil extract or a dry material, which have been presently exemplified.

One aspect of this invention is that cannabis compounds use unextracted natural plant material as a medicine that is both effective at treating symptoms as well as pleasurable to the participant; in this case by providing symptom relief from narcolepsy, and a satisfying organoleptic feel.

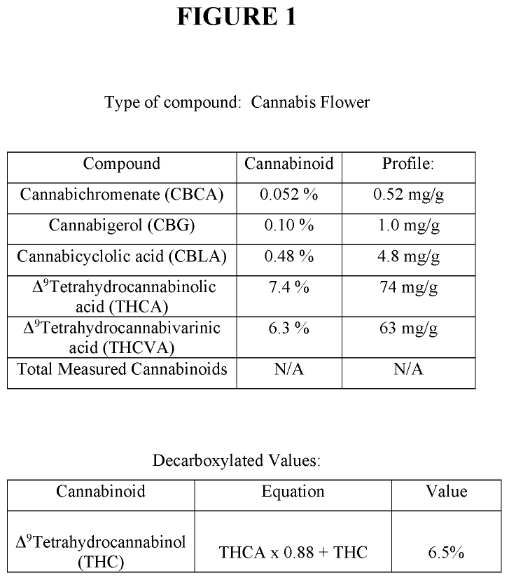

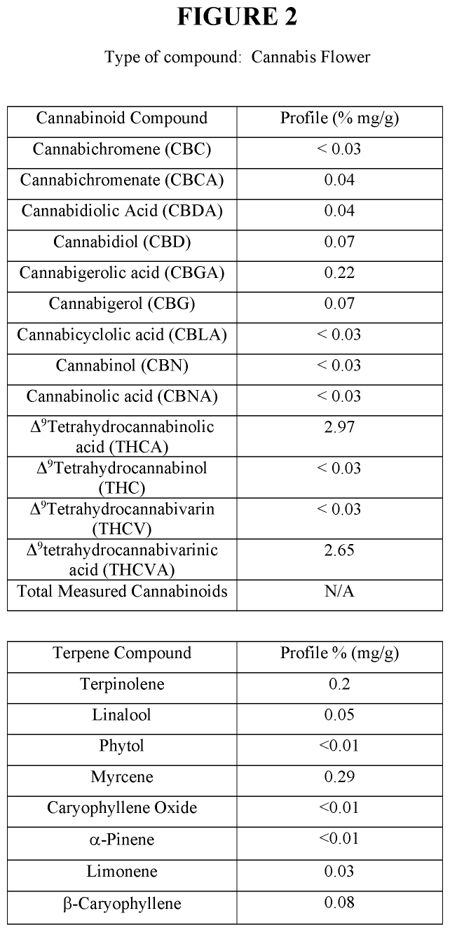

FIGS. 1 and 2 represent cannabis profiles from raw cannabis flowers of a strain that has therapeutic effects in narcolepsy. These two types of raw cannabis flower, or any cannabis having a cannabinoid and/or terpene profile within or near the range of the two types of raw cannabis flower may be substituted into any method of treatment using raw cannabis flower.

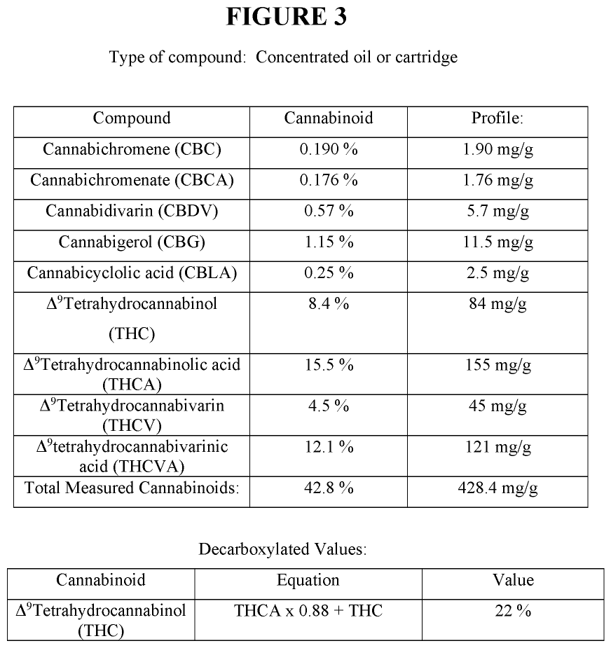

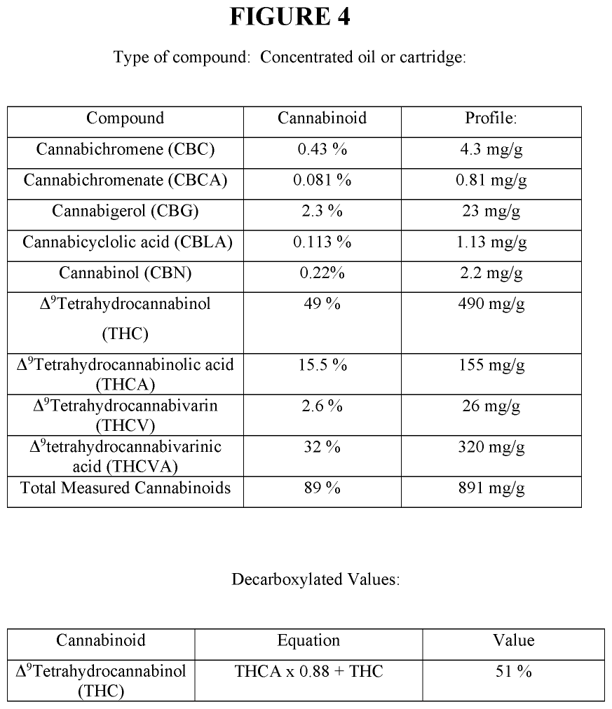

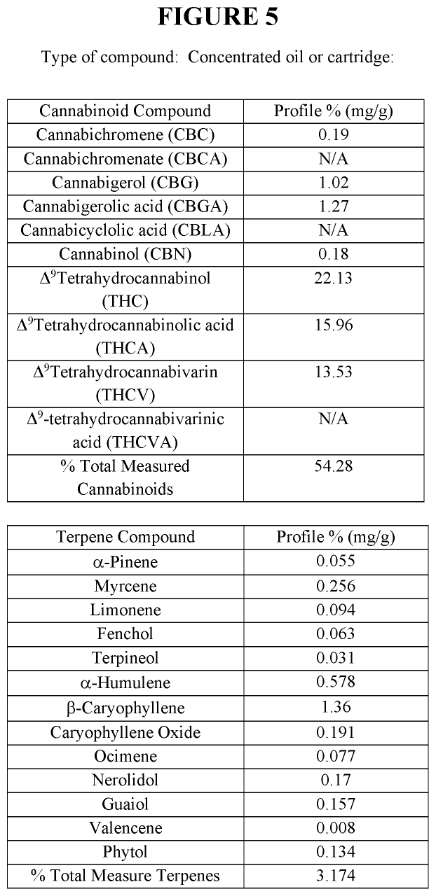

An HPLC analysis of cannabinoid and terpene profiles in cannabis compositions are depicted in FIGS. 3, 4 and 5. These three cannabis compositions, or any cannabis compositions having a cannabinoid and/or terpene profile within or near the ranges of the three cannabis compositions may be used in any method of treatment describe herein using cannabis compositions.

### Methods of Making

Cannabis varieties described herein can be cultivated in any manner known in the art, including commonly known indoor, hydroponic or outdoor growing methods. In a preferred embodiment, a cannabis plant is grown from a clone of a preferred cannabis variety. Cannabis plant parts and/or cannabis flower is harvested from the cannabis plants and dried according to common methods known in the art.

Cannabis extracts herein can be isolated by any manner known in the art.

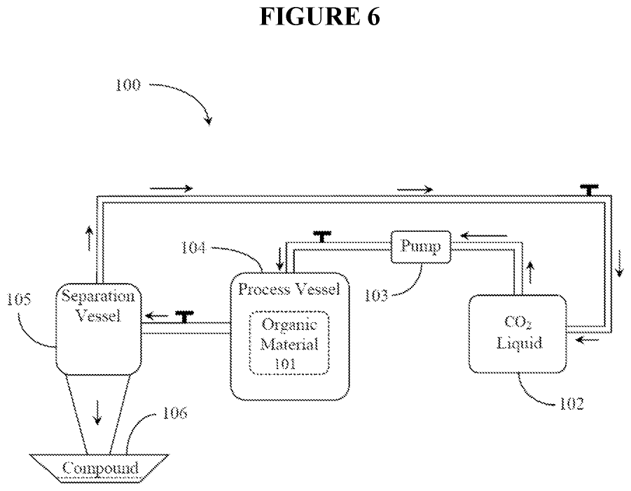

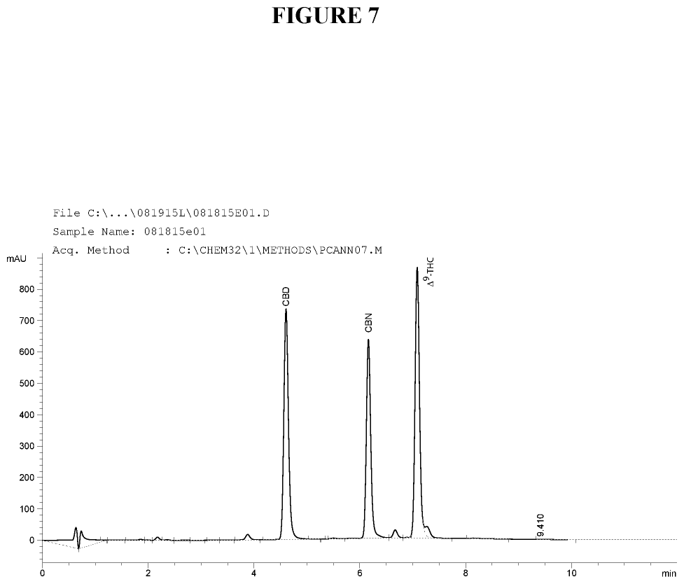

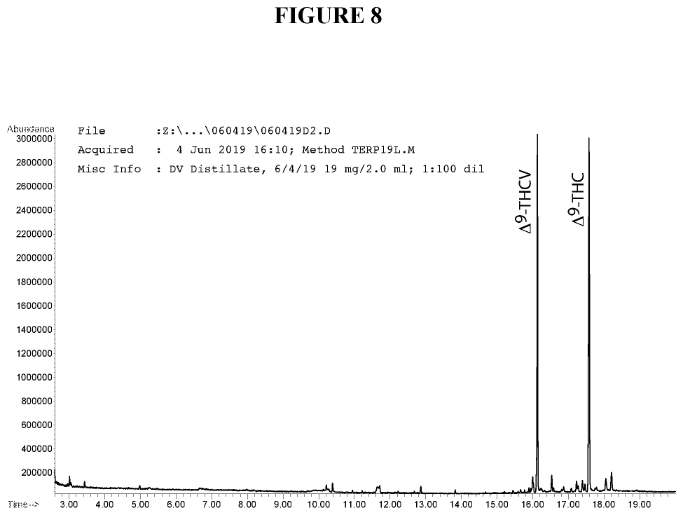

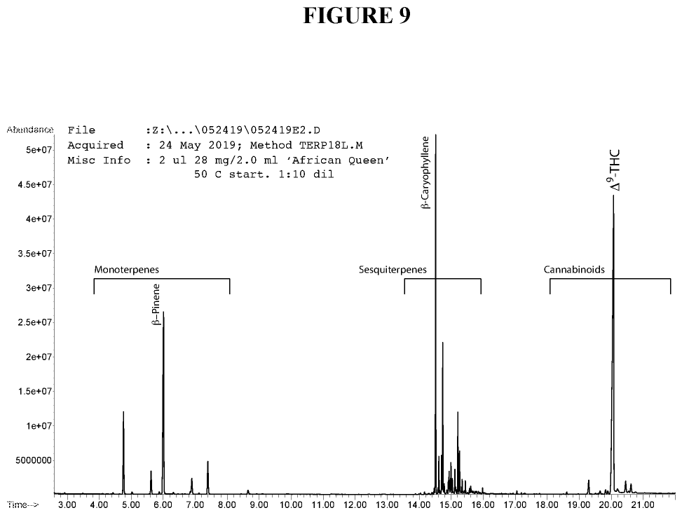

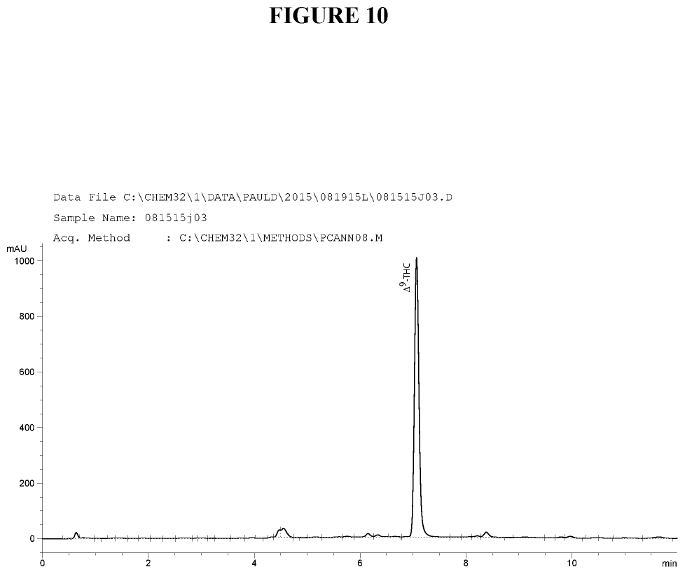

FIG. 6 illustrates a typical extraction process 100 for extracting desired components from a strain of raw cannabis flower. Additional approaches that employ either hydrocarbon solvents (propane, butane, isobutane, pentane, hexane, heptane) or ethanol may also be employed, as described in references below. The process 100 includes components used in a liquid carbon dioxide (CO₂) trichome extraction process. It is important to note herein that the inventors have discovered at least one cannabis strain that may be beneficial to persons with narcolepsy and that includes, before processing, a cannabinoid and terpene profile of identified cannabinoids and terpenes that may be selected targeted via process adjustments for preservation in a final compound. It is also noted herein that the inventors have discovered a final compound for treating narcolepsy in the form of a final product where the product may show efficacy in the treatment of general symptoms of narcolepsy and like or sub-conditions such as cataplexy.

Process 100 represents just one of many possible processes where heating liquid CO₂ to the supercritical conditions is not required. Cannabinoids may have varying points of melt and some processing steps available to the inventor would be damaging to certain cannabinoids targeted for narcolepsy treatment. In general, a raw cannabis of a selected strain and having a certified cannabinoid profile, is placed in a chamber and mixed with a solvent to isolate trichomes that include cannabinoids and terpenes that may be targeted for preservation in a final compound of oil, wax, or paste. In one embodiment, organic material in chamber 101 (broken rectangle) includes at least one Δ⁹tetrahydrocannabinol (THC) and Δ⁹tetrahydrocannabivarin (THCV).

In this embodiment, the cannabinoid profile of the raw product is available to the process engineer and the process engineer may make any necessary process adjustments that may enable the process to be tuned to preserve the at least one Δ⁹tetrahydrocannabinol (THC) and Δ⁹tetrahydrocannabivarin (THCV) present in the raw cannabis cannabinoid profile. At least one cannabinoid THCV in the raw material in chamber 101 is highly desired for preservation in the process to derive a compound oil wax or paste. Such compositions show a surprisingly high activity in treatment of narcolepsy or isolated cataplexy. Said compositions furthermore have shown, according to empirical data, an excellent efficacy in relieving symptoms of both narcolepsy and cataplexy.

In this process, liquid CO₂ from a storage vessel 102 is pumped via pump 103 toward process vessel 104 in the direction of the arrows. Liquid CO₂ may be heated somewhat to a super critical state (with both gaseous and liquid properties) and introduced as a wash for the organic raw material. The CO₂ as an agitated liquid removes the essential components from the raw material and is pumped into a separation vessel 105 to separate the CO₂ from the essential components. The final compound is collected in a collection vessel 106. The recovered CO₂ may be recycled in this process and used again for a subsequent batch. THCV has a similar molecular structure as does the more common cannabinoid THC, both are psychoactive, but

THCV has demonstrated synergistic properties with THC that may show efficacy in the treatment of narcoleptic and or cataplexic symptoms.

### EXAMPLES

### Example 1: 20% Oil (Cannabis Extract)

20% Oil derived from crude CO2 extract and winterization. A suitable product that comprises the desired cannabinoids and terpenes reflective of the initial plant composition can be prepared via a wax and lipid removal technique referred to as “winterization.” In this process crude extract (CO2 extract, or hydrocarbon extract, or ethanol extract) at room temperature is homogenized in 95% ethanol or 100% ethanol using mixing devices such as a glass chemical reactor or a high-shear homogenizer. The resulting suspension has the consistency of pea soup. This suspension is then chilled to−20° C. or preferably −40° C. to coagulate fats and waxes, for a period of at least 12 hours, or preferably 24 hours. The cold suspension is then filtered, the filtrate replaced in −20° C. or preferably −40° C. cold rooms for a minimum of two hours, and filtered a second time. Deposits on filters may be resuspended in ethanol to recover as much as 15 to 20% additional cannabinoid and terpene. Alternatively, the initial chilled suspension can be centrifuged prior to filtration to concentrate the bulk of the waxy material.

Following bulk wax removal, the ethanol may be evaporated from the de-waxed solution, producing a viscous oil suitable for direct incorporation into dosage forms (pills, topical ointments and salves, vape pens, etc.), for a product that ranges from 35% to 65% total cannabinoids and a full terpene profile reflective of the starting plant material. If colored material affects the appearance of the product, the de-waxed alcohol solution may be treated with activated carbon, at a rate of one to five percent, by stirring at room temperature (23° C.), or at somewhat elevated temperature (<50° C.) prior to filtration and ethanol evaporation. If ethanol content of the final product must be controlled, the viscous oil may be passed through a wiped-film evaporator at no less than 150 mTorr pressure and 120° C., to preserve terpene content.

### Example 2: 80% Distillate (Cannabis Extract)

80% Distillate that separates desired cannabinoids from terpenes and other undesired constituents including dark pigments, sugars and gums, can be performed on the viscous, winterized oil described above, via ultra-low-pressure distillation. The oil may require additional treatment prior to distillation to remove polar compounds that interfere with this subsequent processing. For this, the oil is dissolved in a non-polar solvent (preferably pentane, hexane, or heptane) at a 1:2 to 1:4 oil:solvent ratio. This solution is then shaken, in turn, with brine solution at pH 10 (preferably sodium bicarbonate), pH 7 (no additives), and pH 3 (preferably citric acid), dried with a suitable dehydrating agent such as anhydrous sodium sulfate, filtered, and the solvent removed in a suitable evaporator. This degummed oil may also have interfering colored or other contaminating materials removed by suspension in a suitable solvent (preferably hexane, heptane), and passage through a sorbent column filled with granular silicas, aluminas, magnesium silicates, removing interfering compounds. The solvent is again removed by evaporation, and the remaining oil is transferred to a distillation device.

Ultra-low-pressure distillation (<0.1 Torr to <10 mTorr) may be performed in a variety of apparatus, including short-path “pot” stills, Kugelrohr devices (rotating horizontal distillation), wiped-film stills and spinning band stills. In all cases terpenes must be removed in a first “pass” through the apparatus, as the vapor pressure of residual terpenes is great enough to prevent adequately low pressures for cannabinoid distillation at appropriately low temperatures to preserve structure. The terpene removal pass is typically performed at 0.100 to 0.150 Torr, at temperatures below 145° C. Depending on the still design, this initial pass is followed by removal of the oil to a second still, or cleaning of the initial still to remove traces of terpenes.

The second “pass” is performed at much lower pressures (typically 1 to 7 mTorr), and slightly elevated temperatures (165° to 185° C.). Pressures are lowered through use of boosting vacuum pumps, preferably oil diffusion pumps, placed between the distillation apparatus and main mechanical pumps. Under these conditions cannabinoids smoothly distill from heavy, often darkly pigmented “bottoms;” these mixtures are transparent, ranging in color from light amber to colorless, and highly viscous. Distilled oil may be incorporated into any of the above dosage forms, although as terpenes have been removed, cannabis terpenes isolated independently may be added for flavor (e.g.: in vape pens), or for a specific pharmacological effects.

### Example 3: Tinctures (Cannabis Compositions)

Tinctures may be blended with suitable carriers, including alcohol or plant oil carriers.

In one example, an 88% total cannabinoid distillate (31% THCV, 52% THC, 3.4% CBG and minors) is blended to produce a medium chain triglyceride (MCT) coconut-oil based tincture at 8 mg/ml THCV and 13 mg/ml THC. 1.29 g of distillate is weighed into a 100 ml beaker, and 20 ml of MCT oil is added. The beaker is gently warmed over a heat gun while the distillated is stirred into the MCT oil with a glass rod. Once the suspension is entirely uniform, it is poured into a volumetric flask (50.0 mL). The beaker is rinsed with fresh MCT oil two additional times, and these rinsates added to the volumetric flask; the latter flask is brought to volume after inversions to mix. The finished tincture is transparent, without cloudiness, and stable at room temperature for an extended period.

### Example 4: Method of Decarboxylation (Conversion of THCA into THC and/or THCVA into THCV)

THCA and THCVA have useful medicinal properties, but may require decarboxylation to THC and THCV prior to use, particularly if the dosage form is taken by mouth or used topically. The acid forms release a CO2 molecule on heating, which is accomplished through smoking and potentially vaporization routes. The acid forms when highly purified also tend to be crystalline solids, that may or may not be desirable in preparation of finished dosage forms. Decarboxylation can be accomplished by gently heating (100° to 120° C.) at a number of different points in processing. Harvested plant material can be heated in vented ovens, oils can be stirred in beakers on hot plates until visible bubbling stops, and oils can also be decarboxylated in steam autoclaves at 120° C. for 45 minutes. As terpenes can volatilize during any or all of these processes, the engineer must apply appropriate analytical tests to preserve desired finished profiles, and adjust conditions accordingly.

Additional methods of making useful cannabis extracts can be found at: Rosenthal, E. and Zeman, G. 2018. Beyond Buds, Next Generation: Marijuana concentrates and Cannabis Infusions. Quick American Archives. 320 pp, PCT Publication Number WO2004/026857 A2, each of which is hereby incorporated by reference into this application.

### Analytical Methods

Chemical analyses of the cannabis compositions and flowers of the present invention were carried out using standard chemical separation techniques well known to those skilled in the art. Qualitative identification of cannabinoids and terpenes was carried out by GCMS. Quantitative analysis was done by Gas Chromatography (“GC”), with either mass spectrometric (MS) or FID detection, and/or HPLC-PDA (Photo Diode Array). The assays for cannabinoids included orthogonal methods of GC-FID and HPLC for the highest level of accuracy.

Samples were prepared by grinding ^{\tilde{ }}5 g of dried cannabis flower material in a coffee grinder. From this homogenized material, 500±20 mg was placed in a bead beater vial, or sonicated, with ^{\tilde{ }}1 g of 2 mm beads and 5 mL of working solution. Each sample was placed in the bead beater (BioSpec Products Inc.) and homogenized on high for 3 minutes. The vials were centrifuged at 1350×g, decanted into 50 mL falcon tubes, and the process was repeated with fresh working solution. After the second extraction the caps were removed, the vials were decanted into the appropriate falcon tubes, and the vials were rinsed into the falcon tubes with an additional 5 mL of working solution. For samples suspected of having lower concentrations of analytes (i.e. <10% THC or total terpene content ⁻0.5%), 3 mL portions of working solution could be employed. Approximately 2 mL of the extracts were placed in 2 mL centrifuge tubes, and the vials were centrifuged at 9500×g for 5 minutes. The supernatant was placed in a GC vial for terpene analysis without dilution. The supernatant was also diluted with working solution for GC and HPLC analysis. A 1:40 dilution provided the appropriate concentration for analysis of cannabinoids present at concentrations above 1.5%, while a 1:3 dilution allowed for analysis of cannabinoids below this level.

Terpenoids by Gas Chromatography-Flame Ionization Detector (GC-FID)

Terpenes were quantified by a method developed on a GC/MS instrument from Hewlett Packard/Agilent. This method separates and quantifies 17 different terpenoids commonly found in cannabis plant tissue. The terpenoids are each quantified by their own individual calibration curves generated with analytical reference standards (Sigma Aldrich) and all use n-nonane as the internal standard.

The instrumentation includes an HP 6890 (GC) equipped with an autosampler, an HP Ultra-5 column, 50 m length, 0.20 mm internal diameter, 0.25 μm thickness film diameter), and a 5973 Mass selective detector (MSD). The system was controlled with Agilent MSD ChemStation, rev. D.01.02.

Calibration curves were generated by injecting each standard in triplicate and the RSDs provided the measure of precision while the absolute accuracy was determined by comparing the concentrations of the standards predicted by the calibration curve to their “known” values determined by dilution ratios. AOAC International standards for accuracy and precision were used as quality guidelines for every calibration. Check standards were run at the start, middle, and end of every analysis, and recalibration was performed when they varied more than +/−5% of their initial average response. Levels that failed the acceptance criteria and analytes were not quantified at those levels until recalibration of the instrument corrected the deficiency. Most of the curves were linear to nearly two orders of magnitude and based on the sample mass extracted (500 mg) and the two possible extraction volumes (3×3 mL or 3×5 mL), this provided quantitation of terpene levels from 0.01-0.9% or 0.02-1.5% (typical) in the plant matrix.

### Cannabinoids by GC-MS

Cannabinoids were quantified by an analytical method developed and run on the above described GC/MS instrument. This method was developed to separate six neutral cannabinoids, CBD, CBG, CBN, THC, Δ8-THC, and CBC, with the addition of THCV. The cannabinoids are each quantified by their own individual calibration curves generated with analytical reference standards (Restek) and all use phencyclidine as the internal standard.

There was no need to consider chromatographic separation of acidic forms of the cannabinoids due to their immediate conversion to neutral form in the heated injector of the instrument, although a thorough study of the conversion efficiency of THCA was performed and is discussed in section iv. (orthogonal analyses of all samples). We are developing a derivitization method for detection of the acidic cannabinoids as trimethylsilylethers, by GC-MS

Calibration curves were generated by injecting each standard in triplicate and the RSDs provided the measure of precision while the absolute accuracy was determined by comparing the concentrations of the standards predicted by the calibration curve to their “known” values determined by dilution ratios. AOAC International standards for accuracy and precision were used as quality guidelines for every calibration. Check standards were run at the start, middle, and end of every analysis, and recalibration was performed when they varied more than +/−5% of their initial average response. Levels that failed the acceptance criteria and analytes were not quantified at those levels until recalibration of the instrument corrected the deficiency. Due to the very linear nature of the MSD detector, the GC-MS cannabinoid assay generally provided satisfactory results over nearly two orders of magnitude (up to 1.0 mg/mL), however in order to use the same calibration solutions and “validation” procedures for both GC and HPLC the range was reduced to that of the HPLC method. Based on the sample mass extracted (500 mg) and a 3×3 mL extraction (low oil samples), a 1:3 dilution provided quantitation of cannabinoid levels from 0.09-1.35% and the 1:40 dilution from 1.15-18% in the plant matrix. A 3×5 mL extraction (high oil samples, typical), a 1:3 dilution provided quantitation of cannabinoid levels from 0.14-2.25% and the 1:40 dilution from 1.9-30% in the plant matrix.

### Cannabinoids by High Performance Liquid Chromatography-Dual Wavelength UV Detector (HPLC-UV)

An HPLC-UV (also known simply HPLC) assay was developed as an orthogonal method to GC-FID for cannabinoid analyses. This method quantifies six neutral cannabinoids (CBD, CBG, CBN, THC, Δ8-THC, and CBC) as well as THCA and THCVA based on calibration curves generated with analytical standards and an internal reference standard (ibuprofen).

All HPLC analyses were performed using an HP/Agilent 1100 HPLC system comprised of a binary pump, a solvent manager, and an UHPLC autosampler. UV data was collected at 228 nm and 280 nm with a dual-wavelength UHPLC detector. Chromatography was performed on a Restek Raptor C18 column (, 2.7 μm, 3.0×150 mm). HPLC system control, data acquisition and analyses were performed with Agilent Chemstation software.

Calibration was achieved by performing a five-point calibration curve (0.016-0.25 mg/mL for each analyte) followed by linear regression analysis. This analysis was performed with Agilent Chemstation software. The calibration curves were generated by injecting each standard in triplicate and the RSDs provided the measure of precision while the absolute accuracy was determined by comparing the concentrations of the standards predicted by the calibration curve to their “known” values determined by dilution ratios. AOAC International standards for accuracy and precision were used as quality guidelines for every calibration. Check standards were run at the start, middle, and end of every analysis, and recalibration was performed when they varied more than +/−5% of their initial average response.

### Orthogonal Analyses of All Samples

The cannabinoid content was quantified by both GC-MS and HPLC. The main difference between GC and HPLC is that GC involves thermal stress and mainly resolves analytes by boiling points while HPLC does not involve heat and mainly resolves analytes by polarity. There are several reasons that this orthogonal approach to analyses is desirable for highly accurate and reproducible results in determining chemotype. The first reason is related to the difference between the cannabinoids produced naturally by the plant (the acidic cannabinoids) and those that are bioactive (the neutral cannabinoids). Cannabis biosynthesizes all the cannabinoids in their relatively unstable acidic forms, and these forms are generally not bioactive in the traditional sense. The application of heat (flame, vaporizer, oven, etc.) causes a loss of the carboxylic acid group and generates the neutral forms of the cannabinoids, which are generally the bioactive forms that are sought after, however this process is highly variable and not quantitative. If one wants to know the native phytochemical profile of the plant then HPLC should be used since this assay does not involve heat. If one wants to know the possible available amount of bioactive cannabinoids, then GC should be used since conversion to these forms in the injector of the GC is an inherent part of the analytical method.

The second reason is also related to the difference between the acidic and neutral cannabinoids, but has to do with the availability of analytical standards to calibrate the instruments. While all of the neutral cannabinoids (THC, CBG, CBC, CBD, and CBN) are available as analytical standards, THCA is the only acidic cannabinoid available as an analytical standard and the instruments were only calibrated for quantification using actual analytical standards. Technically the HPLC assay could characterize the naturally occurring chemotypes, but the acidic analytes are not available as standards, so this quantification is approximate and considered for information only. The acidic analytes are all quantified by reference to the calibration curve for THCA, and this is not an unreasonable assumption as many of them have approximately the same spectral properties. The GC assay is calibrated with analytical standards, but these are the neutral cannabinoids and their formation from the naturally occurring acidic cannabinoids in the GC injector is not quantitative, which complicates exact characterization of the naturally occurring chemotype.

The final reason is simply to have an internal crosscheck of our results by using orthogonal testing methods. Each type of assay (GC and HPLC) has its strengths and weaknesses, and by using both methods one can compare results and ensure that both the identification and quantitation of the components are accurate. A caveat to this, as mentioned above, is that the conversion of the acidic forms to the neutral forms is not quantitative due to thermal degradation. Under the highly optimized conditions of a GC injector we have found conversion can vary between 75-85% (for analytical THCA standards), while cannabis samples generally have a conversion of 70-80%. Similar conversion rates are also described in literature for highly optimized analytical instruments (Dussy et al. 2004). Because of this incomplete conversion our GC results are consistently 20-30% lower than the HPLC results for cannabis samples. This same conversion efficiency can be applied to estimate the maximum availability of THC based on THCA content when smoking or vaporizing cannabis.

### v. Method “Validation”

In order to demonstrate the performance of a method of analysis, a systematic process known and method validation can be carried out. This process demonstrates the method is fit for its intended purpose and is necessary for the confident use of that method, providing assurance that the results that are reported are precise, accurate, and reflective of the sample. Very few labs in the cannabis industry attempt to validate their assays and this fact, combined with inappropriate sampling have resulted in erroneous data for several varieties. In order to validate the analytical methods employed for this project, an abbreviated protocol similar to Single

Laboratory Validation (SLV) was carried out. Assay “validation” was carried out by spiking blank matrix with the analytes at low, med, and high concentrations and carrying out the assay procedure in replicate (n=5). While some analytes provided better results than others the analyte RSDs, recoveries, and precisions at these concentrations satisfied AOAC guidance (based on mg/mL). In general the RSDs for the terpenes at the low, medium, and high concentrations (varied by terpene but generally 0.016, 0.125, and 1.0 mg/mL) were less than 5%, 4%, and 3% respectively. The absolute bias for these analytes was generally less than 10%, 4%, and 2%. In general the RSDs for the cannabinoids by both GC and HPLC at the low, medium, and high concentrations (0.016, 0.61, and 0.250 mg/mL) were less than 2%, 2%, and 1% respectively. The absolute bias for these analytes was generally less than 10%, 2%, and 2%. The assays all provided satisfactory S/N ratios at the lowest level and this was initially taken as the LOQ. After subsequent re-calibrations (n=3 at each level), the LOQ was taken as the lowest level of the calibration curve that provided acceptable accuracy (<10% error) determined by comparing the known concentration levels (determined by dilution ratios) to the predicted levels (obtained from the signal and calibration curve). The error between the known and measured values establishes the accuracy of the method and verifies that real samples do not present any matrix effects that influence the resulting measurements. The precision, or closeness of individual measurements, of the method is also determined by carrying out all analyses in replicate (n=5). Guidance for acceptable values was taken from publications provided by the AOAC.

The validation revealed that the above-described chemical analysis methods were accurate and reliable, and the use of orthogonal methods of analyses provided an internal check on the assays as well as an understanding of the use of GC to analyze thermally unstable molecules. Using multiple dilution ratios kept samples in the linear ranges of the assays, and method validation verified that precise and accurate results were obtained.

### Volunteer Studies

Inventors shall memorialize findings and claims with further studies. An exemplary study is described below. Volunteers used for this study will be screened via a questionnaire to determine their type of narcolepsy, and severity of narcolepsy symptoms. Volunteers will be chosen according to the type of narcolepsy diagnosed Type 1 (diagnosis code G47.411) or Type 2 (diagnosis code G47.419)) and the severity of symptoms (indicating either severe narcolepsy or mild narcolepsy). Volunteers will be divided into different test groups according to these criteria. Optimal volunteers in each test group will have the same type of narcolepsy, and similar frequency of onset of symptoms. A multi-day baseline assessment period without any treatment will be conducted prior to the randomized study to obtain baseline information about narcoleptic symptoms from each participant. Volunteers will then be randomized and provided with experimental treatments including various cannabis compositions in various medicine combinations. Volunteers will also be assigned to receive placebos, including complete placebos (no active ingredient), a placebo containing no cannabinoids and only terpenes, and placebos containing no terpenes and only cannabinoids. This approach will establish not only efficacy of the cannabinoids and/or terpenes, but also the synergy among the active compounds inherent in each cannabis line used.

As is common to other narcolepsy studies, treatments will be compared using volunteers diary self-assessments scoring frequency, severity, and type of symptoms, as well as overall quality of life assessment of symptoms. Particular emphasis will be placed on number, severity, and as quality of life scores, comparing oral and inhalatory routes of each treatment. The effectiveness of THCV and THC cannabinoids on treating the symptoms of the volunteers.

Some studies described herein, would be a ‘double-blind’ study where the investigator and volunteers would be ‘blinded’ to treatment or placebo group.

In one sample study, five volunteers used two cannabis compositions described herein, which were administered via inhalation. In this sample study, each of the five volunteers has been diagnosed with Type 1 narcolepsy, having symptoms of daytime sleepiness, lethargy and drowsiness during the daytime and disruptive sleep at night. Each volunteer compared the 28% winterized oil of Example 1, and the 40% distillate of Example 2 on an as needed basis by using vape pens. Volunteers self-medicated to alleviate symptoms and reported inhaling the vape pen between 3 and 20 times per day. Each volunteer experienced a minimization of reported narcoleptic symptoms (i.e. no drowsiness, and an alert feeling). None of the volunteers reported side effects of irritability and nervousness, shakiness, disturbances in heart rhythm, and nighttime sleep disruption, commonly associated with other treatments for narcolepsy, such as amphetamine stimulants. On the contrary, each volunteer reported improved sleeping at night, which may have contributed to the success. Volunteers were also asked to compare the 28% cannabis extract and the 40% distillate. Counterintuitively, volunteers used the 40% distillate more frequently to treat symptoms, between 10 and 20 times per day, whereas they used the 20% distillate between 3 and 15 times per day. The 40% distillate was also reported to be more effective at abating reported narcoleptic symptoms.

In some studies, two separate groups of volunteers may be evaluated: one composed of novice cannabis users and one composed of experienced cannabis users. It is helpful to know the past cannabis use history of volunteers since tolerance can occur in experienced users, who will therefore experience the therapeutic effects of the cannabis extracts, cannabis compositions, or cannabis flower described herein, differently than those with no tolerance. However, the rate and duration of tolerance varies with the different effects; a particular individual may have developed tolerance to one cannabis agent but not to another. This may actually serve to increase the therapeutic margin. For instance, tolerance to cognitive and psychomotor impairment, the psychological high, tachycardia, and orthostatic hypertension, tends to develop rather quickly and chronic users may not experience these side effects, while still benefitting from the analgesic or other therapeutic effects of cannabis. Conversely, the novice user who has no tolerance, can be slowly subjected to dose escalation (e.g. over 30 days or more) to build tolerance to these effects before given therapeutic doses. Many times the dysphoria experienced by naive users is enough to cause discontinuation of the treatment, and slow dose escalation which helps induce tolerance to the detrimental side effects may alleviate this.

The biodistribution and PK of the cannabis active agents administered either orally or through inhalation differ substantially. An acute condition may respond better to an inhaled formulation while a chronic condition may respond better to the prolonged plasma concentrations resulting from oral administration. The higher levels of THC and/or THCV metabolites formed from first-pass metabolism after oral formulation administration, which is more potent and has better blood brain barrier penetration than the parent compound, has implications for neurological conditions. The dosing studies described herein evaluate the effects of various doses of the multiplexed cannabis formulations when administered either orally or through inhalation.

Volunteer Sub-Groups and Controls. Large volunteer groups (75-100 volunteers) are studied to evaluate the subjective effects of the cannabis extracts, cannabis compositions, or cannabis flower described herein. For all studies, volunteer groups are chosen from several locations and/or solicited, if drug-naive volunteers are difficult to find. These volunteers are subdivided into experienced and novice cannabis users, and then if the clinical indication warrants it, further subdivided into those receiving either the oral and inhaled formulations. Due to the extremely variable bioavailability, dosage regimens are tailored to the indication and the volunteer. All studies are done with the appropriate medical and/or psychological supervision and evaluation. There are several placebo groups, with the volunteers receiving either complete placebos, a placebo containing no cannabinoids and only terpenes, and placebos containing no terpenes and only cannabinoids. This will serve to establish not only efficacy of the cannabinoids and/or terpenes, but also the synergy. The complete placebo is generated from fats and waxes resulting from cannabinoid extraction and is spiked with terpenes fortifiers for exact and reproducible levels of terpenes to make the placebo without cannabinoids, or it is spiked with cannabinoid fortifiers to make exact and reproducible levels of cannabinoids without the terpenes. Cannabis treatments for these studies will include inhaled, oral buccal, or ingested cannabis. In some embodiments, the inhaled cannabis extracts, cannabis compositions, or cannabis flower are of the present invention. In other embodiments, the inhaled cannabis extracts, cannabis compositions, or cannabis flower of the present invention. In other embodiments, the oral dose of cannabis is prepared from extracts of the specialty cannabis of the present invention.

In one embodiment of this invention the cannabis extracts, cannabis compositions, or cannabis flower of the present invention are tailored to treat symptoms of narcolepsy, isolated cataplexy, sleep apnea, and related disorders, among others. Effectiveness of the treatment will be confirmed by conducting a trial using double blind, randomized treatments comparing the effects of cannabis extracts, cannabis compositions, or cannabis flower containing THCV and/or THC, or combinations of other cannabinoid variants, and/or a combination of various terpenes. Concentrations used for this study may be (2 mg, 5 mg, 10 mg, 15 mg, 20 mg or more THCV) and/or (2 mg, 5 mg, 10 mg, 15 mg, 20 mg or more THC) alone, or in combination with terpenes such as myrcene, limonene, pinene, and/or linalool fortifiers. In some embodiments, the THC:THCV ratio of the cannabis extracts, cannabis compositions, or cannabis flower will be selected from one or more of the ratios described herein. Treatments will be administered via oral or inhaled routes. Dosage levels will be determined based as stated below, or by individually tailoring doses up to the level at which mood improvement is obtained.

Volunteers may also be screened to determine eligibility during their first visit at which baseline pain assessments will be made prior to randomizing subjects into each treatment. Volunteers will also be assigned to receive placebos, including complete placebos (no active ingredient), a placebo containing no cannabinoids and only terpenes, and placebos containing no terpenes and only cannabinoids. This approach will establish not only efficacy of the cannabinoids and/or terpenes, but also the synergy among the active compounds inherent in each cannabis composition, cannabis flower, and cannabis extracts.

Some studies may also compare affects cannabis extracts, cannabis compositions, or cannabis flower of this invention, against treatment provided cannabis, not described in this invention. These studies may test cannabis having little to no THCV and/or THCVA, but having a similar level of THC and/or THCA as the cannabis extracts, cannabis compositions, or cannabis flower of the present invention.

1. A cannabis extract comprising THC and THCV, wherein said extract is at least 1.0% by weight THC, at least 1.0% by weight THCV, and is formulated to treat sleep disorders.

2. A pharmaceutical composition comprising a cannabis extract of claim 1 and a pharmaceutically acceptable carrier.

3. A pharmaceutical composition of claim 2, wherein the sleep disorder is Type 1 Narcolepsy, Cataplexy, Type 2 Narcolepsy, Sleep Apnea, Circadian Rhythm Disorder or Restless Leg Syndrome.

4. A pharmaceutical composition of claim 2, wherein the ratio of THC to THCV is between 15:1 and 1:3.

5. A pharmaceutical composition of claim 2, wherein the ratio of THC to THCV is approximately 1.4:1.

6. A pharmaceutical composition of claim 2, wherein the pharmaceutical composition is an oil suitable for vaping.

7. A pharmaceutical composition of claim 5, wherein the pharmaceutical composition comprises at least 25% THCV by weight.

8. A pharmaceutical composition of claim 5, wherein the pharmaceutical composition comprises at least 40% THCV by weight.

9. A pharmaceutical composition of claim 7, wherein the pharmaceutical composition is formulated to treat narcoplesy.

10. A pharmaceutical composition of claim 1, wherein the pharmaceutical composition is an oil suitable for vaping, and a patient takes 3-15 doses per day to treat narcolepsy.

11. A pharmaceutical composition of claim 9, wherein a dose is 5 and 20 mg of the vaporized cannabis extract.

12. A cannabis extract of claim 1, wherein the cannabis extract is made from a single hybrid cannabis plant, or an asexual clone of said hybrid cannabis plant, or a plant part, tissue, or cell thereof, that produces flower containing a ratio of THCA and THCVA, wherein the ratio of THCA to THCVA is between 1.5:1 and 1:3.

13. A method of treating, alleviating or reducing at least one symptom of a sleep disorder comprising the steps of:
a. Obtaining cannabis extract comprising THC and THCV, wherein said cannabis extract is at least 1.0% by weight THC, and at least 1.0% by weight THCV.
b. Heating the cannabis extract to create a vapor;
c. Inhaling between 5 and 20 mg of the vaporized cannabis extract, 3 to 20 times per day; and
d. Continuing this treatment on a daily basis to alleviate symptoms.

14. A method of claim 13, wherein the cannabis extract of claim 1 further comprises a pharmaceutically acceptable carrier.

15. A method of claim 14, wherein the cannabis extract comprising at least 25% THCV by weight.

16. A method of claim 14, wherein the cannabis extract comprising at least 40% THCV by weight.

17. A method of claim 14, wherein the sleep disorder is Type 1 Narcolepsy, Cataplexy, Type 2 Narcolepsy, Sleep Apnea, Circadian Rhythm Disorder or Restless Leg Syndrome.

18. A method of claim 14, wherein the sleep disorder is narcoplesy.

19. A cannabis composition comprising at least one cannabinoid and at least one terpene for use in a method of treating, alleviating or reducing at least one symptom of a condition selected from the group consisting of narcolepsy, isolated cataplexy, sleep apnea, circadian rhythm sleep disorders and restless leg syndrome, wherein (a) said composition is derived from at least one of a cannabis plant enriched in THCV and THC, wherein the amounts of THC and THCV are substantially equal, (b) wherein at least one cannabinoid is selected from THC, and THCV, and (c) wherein said at least one terpene is selected from a group consisting of camphene, carene, β-caryophyllene, caryophyllene oxide, fenchol, gaiol, α-humulene, limonene, linalool, myrcene, nerolidol, β-ocimene, α-phelladrene, phytol, α-pinene, β-pinene, pinoline, γ-terpinene, α-terpinene, α-terpineol, and terpinolene.

20. A cannabis composition of claim 18, wherein the composition comprises at least 25% THCV by weight.
